(19) United States
(12) Patent Application Publication
(10) Pub. No.: US 2026/0048090 A1
(43) Pub. Date: Feb. 19, 2026

(54) M13 bacteriophages displaying peptide motifs targeting amyloid-beta, methods and uses thereof

(71) Applicants: UNIVERSIDADE DO MINHO, Braga (PT); UNIVERSITY OF AMSTERDAM, Amsterdam (NL)

(72) Inventors: Ivone Marisa PEREIRA MARTINS, Braga (PT); Helmut WILHELMUS HUBERTUS GERARDUS KESSELS, Amsterdam (NL); Joana Cecília VALENTE DE RODRIGUES AZEREDO, Braga (PT); Cláudio Emanuel MOREIRA GOMES, AGUALVA-CACÉM (PT); Joana Margarida LOPES DA SILVA CRISTÓVÃO, OEIRAS (PT)

(73) Assignees: UNIVERSIDADE DO MINHO, Braga (PT); UNIVERSITY OF AMSTERDAM, Amsterdam (NL)

(21) Appl. No.: 19/103,098

(22) PCT Filed: Aug. 14, 2023
(86) PCT No.: PCT/IB2023/058173

§ 371 Date: Feb. 11, 2025

(30) Foreign Application Priority Data
Aug. 12, 2022 (PT) 118159

(51) Int. Cl. A61K 35/76; A61P 25/28; C07K 14/005; C07K 14/47; C12N 7/00; G01N 33/569; G01N 33/68

(52) U.S. Cl. CPC A61K 35/76; A61P 25/28; C07K 14/005; C07K 14/4711; C12N 7/00; G01N 33/56983; G01N 33/6896; C07K 2319/00; C12N 2795/14122; C12N 2795/14133; G01N 2333/005; G01N 2333/4709; G01N 2474/20

(57) ABSTRACT

The present disclosure relates to an engineered M13 bacteriophage displaying amyloidogenic peptide motifs from amyloid beta 42 (Aβ42) at its surface. The present disclosure further relates to the use of the disclosed engineered M13 bacteriophage for detecting early species of Aβ, namely oligomeric and fibrillar Aβ, and preventing its aggregation promoting the inhibition of the progression of Alzheimer's disease and thus contributing to the treatment of this neurodegenerative disorder.

### TECHNICAL FIELD

The present disclosure relates to an engineered M13 bacteriophage, displaying amyloidogenic peptide motifs from the amyloid beta 42 (Aβ42) peptide, at its surface. The present disclosure further relates to the use of the disclosed engineered M13 bacteriophages for detecting early species of amyloid beta (Aβ), namely soluble oligomeric and fibrillar Aβ, and preventing and/or inhibiting its aggregation into fibrils and plaques, promoting the inhibition of the progression of Alzheimer's disease (AD) and thus contributing to the treatment of this neurodegenerative disorder.

### BACKGROUND

Alzheimer's disease (AD) is a chronic, progressive, degenerative brain disorder and the most common type of dementia. The prime suspect of the cause of AD is small sized amyloid beta (Aβ) peptide released by neurons after β and γ secretase cleavage of the amyloid precursor protein (APP), a transmembrane protein abundantly present in the central nervous system (CNS)¹. APP cleavage results in a heterogeneous group of peptides of varying length, being the 40 and 42 amino acid-long isoforms the two main toxic species. Aβ42 has shown to be more prone to aggregation when compared with Aβ40 and a higher presence of Aβ42 has been correlated with higher neurotoxicity. In pathological conditions, Aβ peptides progressively aggregate into soluble oligomers and protofibrils, and deposit as insoluble amyloid plaques², one of the hallmarks of AD. It has become increasingly clear that it is not Aβ immobilized in plaques but instead the still-soluble oligomeric forms that are the toxic species of Aβ³. Even before amyloid plaques can be detected in the brain, Aβ-oligomers can already trigger the loss of synapses⁴, correlating strongly with cognitive decline during early AD. It therefore makes more sense to correlate the level of synapse loss and cognitive decline with the presence of Aβ-oligomers instead of amyloid plaques in the brain. A body of evidence indicated that the majority of toxic species are generated from the secondary nucleation of Aβ monomers on the surfaces of amyloid fibrils that yield Aβ42 oligomers and protofibrils. Therefore, the inhibition of this step is a main target in approaches aimed at limit Aβ aggregation and toxicity. However, currently available immunohistochemical tools are only capable of detecting the presence of amyloid plaques, whereas those able to selectively detect Aβ-oligomers and fibrils in brain samples are lacking.

Peptides hold a great potential for targeting oligomeric and fibrillar Aβ with high affinity and selectivity⁵. However, for use in immunohistochemistry, peptides need to be immunochemically labelled while retaining their architectural structure that confers target recognition and specificity.

Moreover, peptides are not able to cross the blood-brain barrier (BBB), that shelters the central nervous system from the systemic circulation. This bottleneck can be overcome by the use of bacteriophages (phages for short), which are viruses that infect only bacterial cells. It was demonstrated that the filamentous phage M13 is able to cross the BBB. Therefore, the breakthrough of this technology is to engineer M13 to display Aβ-specific peptides.

Phages can be genetically and/or chemically modified to display a variety of biomolecules on their surface, which makes them an attractive biotechnological tool for biomedical research⁶. Because they infect bacteria but not eukaryotic cells, phages are safe for humans and are easy and cheap to produce in large scale.

There is a consensus among researchers that possible treatments to slow or even stop AD progression and preserve brain function may be most effective when administered early in the disease continuum, phages have been extensively used in clinical practice to treat bacterial infections since the 1920's⁷. Notably, filamentous phages such as M13 are generally well tolerated by the immune system⁸ and have the capacity to cross the BBB⁹. In fact, naturally occurring phages can be found at relatively high abundance (up to 10⁴ plaque forming units (pfu)/ml) in the cerebrospinal fluid (CSF) of healthy humans¹⁰. The ability to be recognized by the immune system and to cross the BBB likely depends on the type of phage and the peptide sequence displayed on its surface.

These facts are disclosed in order to illustrate the technical problem addressed by the present disclosure.

### GENERAL DESCRIPTION

The present disclosure relates to an engineered M13 bacteriophage with surface amyloidogenic peptide motifs from Aβ42.

Protein aggregation and toxic deposition of amyloid beta (As) is a hallmark feature in Alzheimer's disease (AD) whose mitigation is critical to delay disease onset.

An aspect of the present disclosure relates to genetic manipulation of M13 bacteriophage to generate synthetic phages that display surface peptide motifs known to recognize Aβ42 conformers via homotypic interactions. Using Aβ42 aggregation kinetics, phages were tested in vitro based on their effects on Aβ42 aggregation and fibrils formation. Based on immunofluorescence analysis, it was observed that two of the engineered phages (Aβ30-39 and Aβ33-42) co-localize with Aβ inclusions found in brain tissue of AD patients and mice models.

Another aspect of the present disclosure relates to the use of engineered phages as an immunohistochemistry tool to detect Aβ oligomers and fibrils in postmortem brain tissue.

In an embodiment, M13 phages were genetically engineered to display, on the surface, small amyloidogenic peptide motifs from Aβ42. These small amyloidogenic peptide motifs from Aβ42 were previously reported to recognize Aβ oligomers and fibrils with nanomolar affinity and are able to neutralize the toxicity of Aβ oligomers when grafted into the complementarity determining regions (CDRs) of antibodies¹¹.

An aspect of the present disclosure relates to M13 bacteriophage comprising, at the surface, amyloidogenic peptide motifs, AB30-39 (SEQ ID No. 1—AIIGLMVGGV) and AB33-42 (SEQ ID No. 2—GLMVGGVVIA), for use in medicine as a medicament, or for use as a dye.

In an embodiment, the bacteriophages are for use in the detection, and/or the diagnostic of amyloid-beta oligomers and amyloid-beta fibrils, and in the prevention, or the inhibition of the aggregation of amyloid-beta 42.

In an embodiment, the engineered (or modified) bacteriophages are for use in the detection, the diagnostic, the prevention, or the treating of the aggregation of amyloid-beta oligomers and amyloid-beta fibrils.

In an embodiment, the engineered (modified) bacteriophages are for use in the detection, the diagnostic, the prevention, or the treating of a neurodegenerative disease.

In an embodiment, the M13 bacteriophage is for detecting or diagnosing the presence of amyloid-beta oligomers and amyloid-beta fibrils in brain tissue sample.

In an embodiment, the bacteriophages are for use in detecting, diagnosing, preventing, or treating Alzheimer's disease or a neurodegenerative disease that is positively influenced by the decrease in aggregation of amyloid-beta oligomers and amyloid-beta fibrils.

Another aspect of the present disclosure relates to a pharmaceutical composition comprising the bacteriophages of the present disclosure.

In an embodiment, the composition of the present disclosure further comprises a suitable pharmaceutical excipient.

In an embodiment, the composition is for use in the diagnostic/prevention of neurodegenerative diseases by intravenous (IV) administration.

In an embodiment, the administration of the composition is in a daily dose to a person with a neurodegenerative disease that is positively influenced by the decrease of the aggregation of amyloid-beta, wherein the dosage amount is less than 10¹⁰ pfu/day. In another embodiment, the administration of the composition is in every two days. In another embodiment, the administration of the composition is in every 3 days. In yet another embodiment, the administration of the composition is in a weekly dose. The administration in all cases can occur during a given period of time.

In an embodiment, the administered dosage amount ranges from 10⁴-10¹⁰ pfu/day, preferably 10⁶-10⁹ pfu/day.

Another aspect of the present invention relates to a kit, for detecting or diagnosing amyloid-beta oligomers and/or amyloid-beta fibrils, comprising the bacteriophages of the present disclosure.

Another aspect of the present invention relates to an in vitro method of detecting or diagnosing the presence of amyloid-beta oligomers and amyloid-beta fibrils.

In an embodiment, the method comprises the steps of:
a) Incubate the brain tissue with the engineered phages of the present disclosure, and wash the excess;
b) Incubate the washed brain tissue with rabbit anti-fd bacteriophage primary antibody, FITC-labelled goat anti-rabbit IgG secondary antibody, and DAPI stain.

In an embodiment, the brain tissue is incubated with 50-100 μl of a solution comprising the phage at a concentration of 10⁴ to 10¹⁰ pfu/ml (in a saline solution of TBS 1× or PBS 1×) overnight at 4° C. in a humidified chamber.

In an embodiment, the washed brain tissue is incubated with 1:1000 diluted rabbit anti-fd phage primary antibody overnight at 4° C. in a humidified chamber. The brain tissue with rabbit anti-fd bacteriophage antibody is washed and thereafter incubated with the FITC-labeled goat anti-rabbit IgG secondary antibody, then washed and stained with DAPI for fluorescence imaging.

In an embodiment, the method of the present disclosure further comprises washing the tissue slide with TBST 1× five times for 10 minutes each time.

In an embodiment, the daily form consists of an intravenous solution, comprising a definitive amount of M13 bacteriophage comprising an amyloidogenic peptide motif (AB30-39, AB33-42 or both), the whole of which is intended to be administered as a single dose, or multiple doses if necessary for a given period of time.

The present disclosure relates to a modified M13 bacteriophage (engineered M13 bacteriophage) comprising, at its surface, amyloidogenic peptide motifs for use as a medicament or therapeutic drug, or for use as a dye, or tag, or label, wherein the peptide motifs comprise at least a sequence 90% identical to the sequences of the following list: SEQ ID No 1, SEQ ID No 2, and mixtures thereof. Preferably 91% identical, 92% identical, 93% identical, 94% identical, 95% identical, 96% identical, 97% identical, 98% identical, 99% identical or 100% identical.

In an embodiment, the bacteriophage comprises a DNA sequence at least 90% identical to SEQ ID No. 5. Preferably 91% identical, 92% identical, 93% identical, 94% identical, 95% identical, 96% identical, 97% identical, 98% identical, 99% identical or 100% identical.

In another embodiment, the bacteriophage comprises a DNA sequence at least 90% identical to SEQ ID No. 6. Preferably 91% identical, 92% identical, 93% identical, 94% identical, 95% identical, 96% identical, 97% identical, 98% identical, 99% identical or 100% identical.

In an embodiment, the peptide motif of the bacteriophage comprises at least a sequence 90% identical to SEQ ID No. 1, preferably 91% identical, 92% identical, 93% identical, 94% identical, 95% identical, 96% identical, 97% identical, 98% identical, 99% identical or 100% identical; and a DNA sequence at least 90% identical to SEQ ID No. 5, preferably 91% identical, 92% identical, 93% identical, 94% identical, 95% identical, 96% identical, 97% identical, 98% identical, 99% identical or 100% identical.

In another embodiment, the peptide motif of the bacteriophage comprises at least a sequence 90% identical to SEQ ID No. 2, preferably 91% identical, 92% identical, 93% identical, 94% identical, 95% identical, 96% identical, 97% identical, 98% identical, 99% identical or 100% identical; and a DNA sequence at least 90% identical to SEQ ID No. 6, preferably 91% identical, 92% identical, 93% identical, 94% identical, 95% identical, 96% identical, 97% identical, 98% identical, 99% identical or 100% identical.

In an embodiment for better results, the bacteriophage comprises, at its surface, five to eight of said amyloidogenic peptide motifs. It was observed that the presence of 5 to 8 peptides at the surface of each phage surprisingly improve the avidity and specificity for oligomers in relation to monomers than individual Aβ-peptides. This, improves the diagnostic and prevention of Alzheimer's disease or a neurodegenerative disease that is positively influenced by the decrease in aggregation of amyloid-beta oligomers and amyloid-beta fibrils.

In an embodiment, the disclosed bacteriophage is for use in the detection, the diagnostic, the prevention, and/or the treatment of the aggregation of amyloid-beta oligomers and/or amyloid-beta fibrils. In a further embodiment, the disclosed bacteriophage is for use in the quantification of the aggregation of amyloid-beta oligomers and/or amyloid-beta fibrils. In an embodiment, the treatment can occur either by the inhibition of aggregation, or disaggregation of the already formed amyloid-beta aggregates.

In another embodiment, the disclosed bacteriophage is for use in the detection, the diagnostic, the prevention, or the treatment of a neurodegenerative disease.

In an embodiment, the disclosed bacteriophage is for use in the detection, quantification, and/or diagnostic of the presence of amyloid-beta oligomers and/or amyloid-beta fibrils in a tissue sample.

In an embodiment, the tissue sample is a brain tissue sample.

In an embodiment, the bacteriophage is for use in the detection, diagnostic, prevention, and/or treatment of Alzheimer's disease or a neurodegenerative disease that is positively influenced by the decrease in aggregation of amyloid-beta oligomers and amyloid-beta fibrils. Surprisingly, the disclosed engineered M13 bacteriophages allows an early detection of Alzheimer's disease, or a neurodegenerative disease that is positively influenced by the decrease in aggregation of amyloid-beta oligomers and amyloid-beta fibrils, since it is able to detect and bind to oligomeric and fibrillar Aβ, which are relevant in the early stages of these diseases.

An aspect of the disclosure relates to a M13 bacteriophage comprising, at its surface, five to eight amyloidogenic peptide motifs wherein the peptide motifs comprise at least a sequence 90% identical to the sequences of the following list: SEQ ID No 1, SEQ ID No 2, and mixtures thereof. Preferably 91% identical, 92% identical, 93% identical, 94% identical, 95% identical, 96% identical, 97% identical, 98% identical, 99% identical or 100% identical.

The present disclosure also relates to a pharmaceutical composition comprising the disclosed bacteriophages.

In an embodiment, the pharmaceutical composition further comprises a suitable pharmaceutical excipient.

In an embodiment, the pharmaceutical composition is for use in the prevention, and/or treatment of Alzheimer's disease or a neurodegenerative disease that is positively influenced by the decrease in aggregation of amyloid-beta oligomers and amyloid-beta fibrils, wherein the composition is in a solution form for intravenous administration.

In an embodiment, the intravenous dosage amount is less than 10¹⁰ pfu/day. In another embodiment, the intravenous dosage amount ranges from 10⁴-10¹⁰ pfu/day.

An aspect of the present disclosure comprises a kit for detecting, quantifying, and/or diagnosing amyloid-beta oligomers and amyloid-beta fibrils, comprising the disclosed bacteriophages. In an embodiment, the kit is for use in the diagnostic and monitorization of Alzheimer's disease.

The present disclosure also relates to a method of detecting or diagnosing the presence of amyloid-beta oligomers and/or amyloid-beta fibrils, in a tissue sample comprising the steps of incubating the tissue sample with a solution comprising the disclosed bacteriophage; detecting the presence of the bacteriophage in the tissue sample.

In an embodiment, the method further comprises a step of incubating the tissue sample with a first antibody suitable to bind to the bacteriophage. In a further embodiment, the first antibody (primary antibody) is a rabbit anti-fd phage antibody.

In an embodiment, the method further comprises a step of incubating the tissue sample with a second antibody suitable to bind to the first antibody, wherein the second antibody (secondary antibody) is a fluorescent antibody, i.e., an antibody that has been tagged with a fluorescent compound.

In an embodiment, the second antibody is a fluorescein isothiocyanate (FITC)-labeled goat anti-rabbit immunoglobulin G (IgG) antibody.

In an embodiment, the tissue sample is a brain tissue sample.

In an embodiment, the concentration of bacteriophage in the solution ranges from 10⁴ pfu/ml-10¹⁰ pfu/ml.

An aspect of the present disclosure relates to the use of a M13 bacteriophage as a dye/tag, or label, wherein said M13 bacteriophage comprises, at its surface, amyloidogenic peptide motifs, wherein the peptide motifs comprise at least a sequence 90% identical to the sequences of the following list: SEQ ID No 1, SEQ ID No 2, and mixtures thereof. Preferably 91% identical, 92% identical, 93% identical, 94% identical, 95% identical, 96% identical, 97% identical, 98% identical, 99% identical or 100% identical.

### BRIEF DESCRIPTION OF THE DRAWINGS

The following figures provide preferred embodiments for illustrating the disclosure and should not be seen as limiting the scope of invention.

FIG. 1 illustrates the process of genetic manipulation of the M13 phage.

FIG. 2 shows an embodiment of the effect of AB-phages on Aβ42 aggregation.

FIG. 3 shows an embodiment of the effect of AB-phages on surface-catalysed secondary nucleation of Aβ342.

FIG. 4 shows an embodiment of the effect of AB-phages on fibril content at the end-point of the aggregation curves.

FIG. 5 shows representative fluorescence images of control and APP mice brain tissue samples in two different age groups: 3-4 months and 9-12 months (FIG. 5A), and signal quantification (FIGS. 5B and C).

FIG. 6 shows representative fluorescence images of control and AD human brain tissue samples and signal quantification.

FIG. 7 shows representative fluorescence images of control and APP mice brain tissue samples with and without antigen retrieval.

FIG. 8 shows representative fluorescence images of M13 and AB33-42 in APP mice brain tissue samples with different fixation times.

### DETAILED DESCRIPTION

The present disclosure relates to an engineered M13 bacteriophage displaying amyloidogenic peptide motifs from amyloid beta 42 (Aβ342) at its surface. The present disclosure further relates to the use of the disclosed engineered M13 bacteriophage for detecting early species of Aβ, namely oligomeric and fibrillar Aβ, and preventing its aggregation promoting the inhibition of the progression of Alzheimer's disease and thus contributing to the treatment of this neurodegenerative disorder.

All reagents were of the highest grade commercially available. Thioflavin T (ThT) was obtained from Sigma. A Chelex resin (Bio-Rad) was used to remove contaminant trace metals from all solutions. Human Aβ42 expression plasmid (pET-Sac-Abeta (M1-42), SEQ ID No. 9) was used to prepare recombinant Aβ42 (SEQ ID No. 8). Recombinant Aβ42 was expressed in Escherichia coli and purified according to Walsh et al.¹². To obtain the monomeric form, 4 mg of Aβ42 was dissolved in 7 M guanidine hydrochloride and eluted in a Superdex S75 (GE Healthcare) with 50 mM HEPES pH 7.4 and used immediately. Amyloid fibrils of Aβ42 were prepared by incubation of 5 μM Aβ42 at 37° C. for 24 hours under quiescent conditions. Low-bind tubes (Axygen Scientific, Corning) were used in all manipulations of Aβ42.

In an embodiment, genetic manipulation of M13 phage was performed.

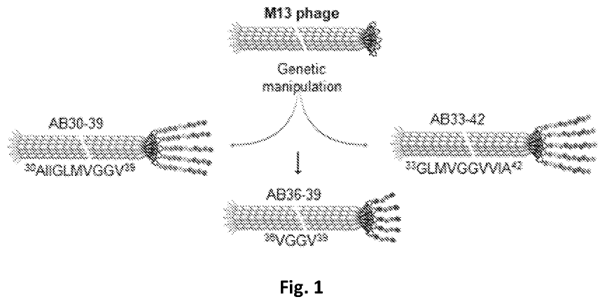

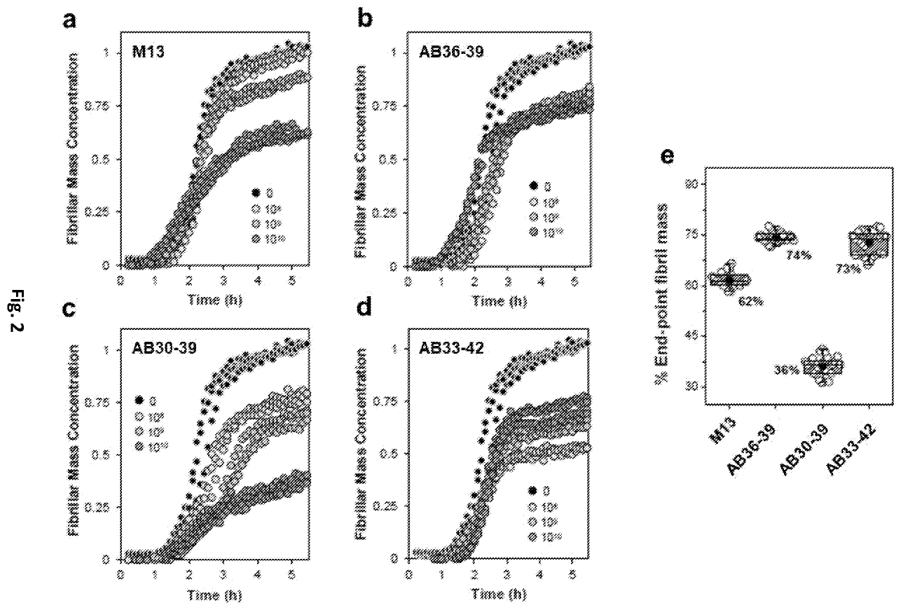

In an embodiment, insert preparation was performed. Two amyloidogenic peptide residues of 10 amino acids (Aβ-based) were primer designed to be cloned into the genome of the M13KE phage. Aβ330-39 (SEQ ID No. 1—AIIGLMVGGV) and Aβ33-42 (SEQ ID No. 2—GLMVGGVVIA) peptide motifs from the Aβ42 peptide were genetically fused to the N-terminus of the gene 3, leading to peptide expression on the coat protein III of the M13 phage (FIG. 1).

FIG. 1 illustrates the process of genetic manipulation of the M13 phage. The 10 amino acid Aβ-based peptide motifs Aβ30-39 (AIIGLMVGGV) and Aβ33-42 (GLMVGGVVIA), were cloned in the gene 3 of M13 with consequent display at the phage surface protein III.

Following the procedure, a third engineered phage was obtained displaying only four amino acids from the Aβ42 peptide-Aβ36-39 (VGGV), because only part of the correct Aβ sequence was inserted into the M13 phage genome. This phage was named Aβ36-39.

In an embodiment, the 10 amino acid Aβ-based peptide motifs Aβ30-39 (AIIGLMVGGV) and Aβ33-42 (GLMVGGVVIA) were cloned in the genome of M13 based on phagemid cloning system¹³. Basic components of a phagemid include the replication origin of a plasmid, the selective marker (usually an antibiotic resistance marker), the intergenic region (IG region, usually contains the packing sequence and replication origin of minus and plus strands), a gene of a phage coat protein, restriction enzymes recognition sites, a promoter and a DNA segment encoding a signal peptide¹³. For the phagemid construction, the commercial plasmid pETDuet-1 (Novagen, Darmstadt, Germany, SEQ ID No. 3) was used. This plasmid contains the ampicillin resistant gene; the T7 promoter (allows gene transcription that affects the expression level of fusion genes); the signal peptide pelB (facilitates the translocation through the bacterial membrane of phage proteins and their assembly in phage particles). The entire sequence of the gene 3 of the M13 phage (codes for the phage coat protein III) was first cloned between the HindIII-NotI restriction sites of MCS-1 (multiple cloning site 1). Then, the pelB sequence was cloned between BamHI-EcoRI also on MCS-1, thus obtaining an intermediate plasmid with SEQ ID No. 4. The Aβ peptide sequences were cloned immediately before the gene 3 of the M13 on the MCS-1 of the pETDuet-1 plasmid between Sall—Sacl restriction sites, resulting in a plasmid with SEQ ID No. 5 (phagemid AB30-39) or SEQ ID No. 6 (phagemid AB33-42). This recombinant plasmid was transformed in E. coli competent cells, positive clones were confirmed by polymerase chain reaction (PCR) and sequencing.

In an embodiment, phage particles were produced. Because phagemids can be converted to phage particles with the same morphology by co-infection with helper phages, the kanamycin resistant M13KO7 helper phage (N0315S, New England BioLabs® Inc), a derivative of the M13 phage containing the kanamycin resistance gene, was used. An infection protocol to merge the gene 3 of the plasmid with the gene 3 of the helper phage, allowing the display of the Aβ sequence in the phage coat protein III, was performed following the protocol from New England Biolabs. Briefly, the cells containing the phagemid were grown in LB medium with ampicillin at a final concentration of 20 mg/ml and infected with 50 μl of the M13KO7 helper phage (10⁸ pfu/ml) at 37° C., 250 rpm for 90 minutes. Then, kanamycin was added to a final concentration of 70 μg/ml, and the solution incubated overnight at 37° C. and 250 rpm. To separate bacteria cells from the phages, centrifugation at 6000 rpm for 10 minutes was performed and the supernatant transferred to a new tube. Phage was precipitated by the addition of PEG/NaCl solution followed by incubation at 4° C. for 2 hours and resuspended in Tris-buffered saline (TBS). Phage genomic ssDNA was isolated using equal volume of phenol-chloroform-isoamyl alcohol (25:24:1, v/v), purified with an equal volume of chloroform, precipitated with 100% ethanol and resuspended in Tris-EDTA (TE).

In an embodiment, to check the Aβ sequences, the DNA of the synthetic phages was sequenced using a primer (SEQ ID No. 7) that reads the region of interest of the gene 3. Phage titration was performed following the double agar overlay technique. Briefly, 10 μl of serially diluted phage, 200 μl of host bacteria culture and 3 ml of soft agar were mixed and poured onto a LB plate with ampicillin. After overnight incubation at 37° C., the plaque forming units (pfus) were determined.

Methods for the alignment of sequences for comparison are well known in the art, such methods include GAP, BESTFIT, BLAST, FASTA and TFASTA. GAP uses the algorithm of Needleman and Wunsch to find the global (over the whole the sequence) alignment of two sequences that maximizes the number of matches and minimizes the number of gaps¹⁴. The BLAST algorithm calculates percent sequence identity and performs a statistical analysis of the similarity between the two sequences¹⁵. The software for performing BLAST analysis is publicly available through the National Centre for Biotechnology Information (NCBI). Global percentages of similarity and identity may also be determined using one of the methods available in the MatGAT software package: an application that generates similarity/identity matrices using protein or DNA sequences¹⁶. Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

In an embodiment, another phage displaying only 4 amino acids peptide was also obtained Aβ36-39 (VGGV, SEQ ID No 10) (FIG. 1).

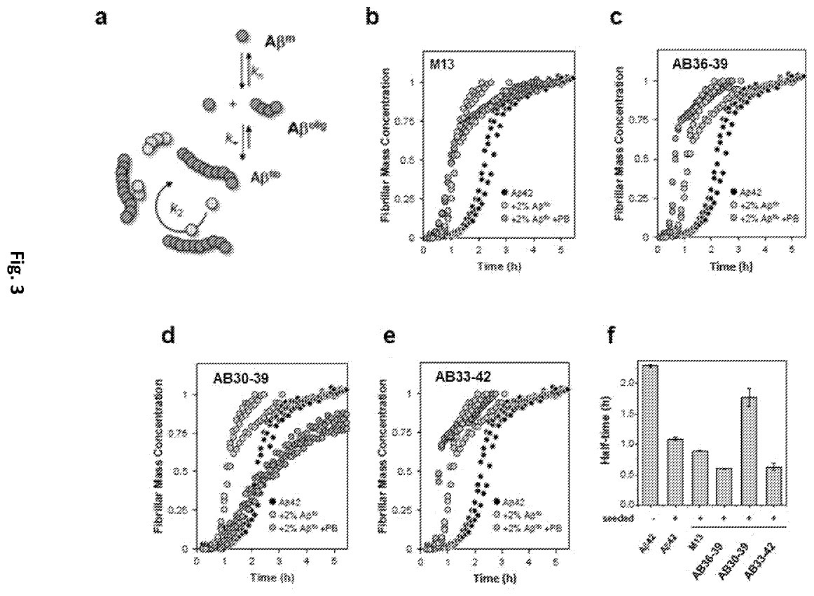

In an embodiment, Aβ42 aggregation kinetics was determined (FIGS. 2 and 3). Aggregation kinetics were determined by recording the ThT fluorescence intensity as a function of time in a plate reader (Fluostar Optima, BMG Labtech) with a 440 nm excitation filter and a 480 nm emission filter, as per original reports. The fluorescence was recorded using bottom optics in half-area 96-well polyethylene glycol-coated black polystyrene plates with a clear bottom (3881, Corning). Aβ42 monomer was isolated by gel filtration (Tricorn Superdex75 column, GE Healthcare) in 50 mM HEPES, pH 7.4. 10 μM of ThT was added to each condition. AB phages and M13 were added at the start of the reaction at different titers (10⁸, 10⁹ and 10¹⁰ pfu/ml). Assays were performed in triplicates at 37° C., without agitation, with fluorescence read every 400 seconds. Data was analyzed using Amylofit and processed in Origin. Fibril mass, estimated from ThT intensity at end points was averaged from fluorescence recordings at the post transition plateau stage.

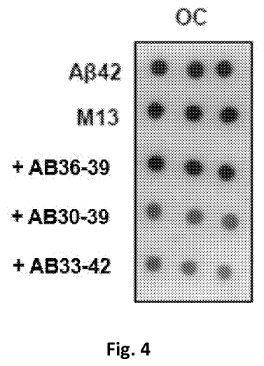

In an embodiment, immunoblot analysis was performed (FIG. 4). Aβ42 aggregates obtained at the plateau phase of each aggregation kinetic curve were diluted up to eight times and dotted in triplicates onto PVDF membrane and probed with a 1:1000 dilution of anti-amyloid fibril OC antibody (Aβ9234, Merck Millipore), according to the manufacturer's instructions.

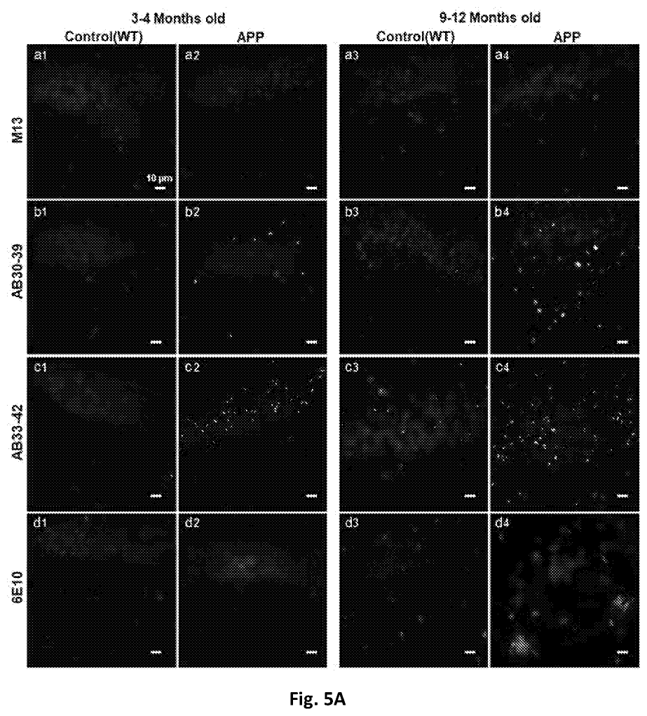

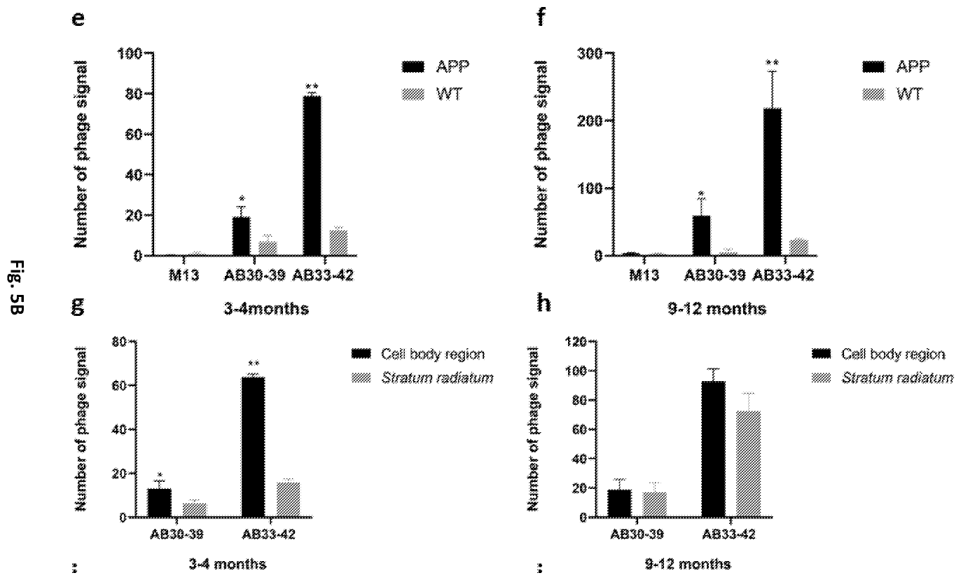

In an embodiment, immunofluorescence assays were performed (FIG. 5 to 8). Brains of age-matched APPswe/PS1dE9 transgenic and wild-type mice were isolated in ice-cold phosphate-buffered saline (PBS) pH 7.45, deep-frozen with liquid nitrogen and stored at −20° C. Brain tissue was sectioned using a cryostat (CM3050S, Leica) with chamber temperature −15° C. and object temperature −13° C. The cutting angle was set to 5 degrees and the size of the sections to 10 μm. The tissue was trimmed to reach the hippocampal region. The sections were mounted on SuperFrost Plus glass (11950657, Thermo Scientific) and stored at −20° C. The same procedure was applied to post-mortem human hippocampal 7 μm sections from AD patients and respective controls (Table 1). Cryopreserved slides were fixated for 10 min with 4% paraformaldehyde (PFA) in PBS. Slides were incubated for 1 hour at room temperature with blocking solution of 2.5% bovine serum albumin (BSA) (Sigma-Aldrich, 10735086001) in TBS plus 0.1% of Tween-20 (TBST). Next, slides were incubated with phages at a concentration of 10⁸ pfu/ml in TBST overnight at 4° C. in a humidified chamber. Slides were washed in TBST five times for 10 minutes and subsequently incubated overnight at 4° C. in a humidified chamber with 1:1000 diluted rabbit anti-fd phage antibody (B7786, Sigma). The slides were then washed and incubated for 2 hours at room temperature with 1:200 diluted FITC-labelled goat anti-rabbit IgG antibody (F9887, Sigma). For 6E10 antibody staining, antigen retrieval was performed by heat-mediated treatment with sodium citrate buffer 10 mM pH 6.0 (51894-500G, Sigma-Aldrich), where a boiling state was set for 20 min followed by a cooling down to room temperature for 20 min. Slides were incubated with 1:5000 diluted mouse anti-β-amyloid 6E10 antibody (80300, BioLegend), followed by 1:700 diluted donkey anti-mouse IgG (H+L) labeled with Alexa Fluor 594 (A-21203, Thermo Scientific). All antibodies were diluted in TBST with 1% BSA. Slides were covered with Vectashield mounting medium with DAPI (VectorLabs). Images were acquired with a fluorescence microscope (Nikon Eclipse E400) and analyzed using ImageJ. The quantification was performed in 40× amplification images using a colour threshold set to identify the fluorescein signal (phages) and all puncta within a defined size threshold between 10-150 pixels were counted.

Table 1: Brain tissue samples from human donors. Thal phase based on the detection of immunopositive amyloid in cortical and subcortical areas: phase (3)—brainstem/midbrain. AD neuropathological change (Neuro Change) was evaluated trough ABC score: A—thal Phase for Aβ plaques; B—Braak neurofibrillary tangles score; C—CERAD neuritic plaque score.

| Sample | Case ID | Sex | Age | Thal Phase | AD Change |
|---|---|---|---|---|---|
| Control | S19-00038 | Female | 76 | — | A₀B₀C₀ |
| | S91-290 | Male | 86 | — | A₁B₁C₀ |
| | S92-301 | Female | 81 | — | A₁B₁C₀ |
| AD patients | S04-00053 | Female | 73 | 3 | A₃B₃C₃ |
| | S09-217 | Female | 89 | 3 | A₃B₂C₂ |
| | S16-170 | Female | 79 | 3 | A₃B₃C₂ |

In an embodiment, parametric data were evaluated by the Student t-test for two-group comparisons, p<0.05 was considered statistically significant for all analyses.

In an embodiment, presence of Aβ peptide motifs on the surface of the M13 phage was determined. Previous studies showed that peptides corresponding to the c-terminal amyloid-beta (Aβ) amino acids 30 to 39 (³⁰AIIGLMVGGV³⁹) and 33 to 42 (³³GLMVGGVVIA⁴²) are reactive to both Aβ-oligomers and fibrils, but only weakly to monomers¹¹. These Aβ peptide motifs were cloned in phagemids to be exposed onto coat protein III of M13 filamentous phage (FIG. 1). By merging these phagemids with a M13 helper phage, they were packaged into phage capsids as ssDNA. These synthetic phage particles, which were named Ab36-39, Ab30-39 and Ab33-42, displayed 5 to 8 copies of peptide motifs Aβ336-39, Aβ30-39 and Aβ33-42, respectively, on their surface, clustered at the end of the filamentous phage (FIG. 1).

In an embodiment, to examine if the engineered phages would inhibit the aggregation of on Aβ42, aggregation kinetics were determined. The aggregation process of Aβ42 leading to fibrils can be monitored using thioflavin T (ThT) fluorescence, which is an amyloid-sensitive dye whose intensity can be correlated to fibril mass. The reaction started from highly homogeneous preparations of monomeric Aβ42¹⁷, allowing to estimate the effect of inhibitors on the reaction rate. ThT fluorescence kinetics of Aβ42 aggregation showed the hallmark sigmoid-shaped curves of Aβ42 aggregation, which reflect the complex kinetics through which Aβ42 monomers self-assemble into oligomers and fibrils (FIG. 2).

In an embodiment, FIG. 2 shows the effect of AB-phages on Aβ42 aggregation. The aggregation of 5 μM Aβ42 was monitored by ThT emission with and without added M13 phage (FIG. 2-a), Ab36-39 (FIG. 2-b), Ab30-39 (FIG. 2c) and Ab33-42 (FIG. 2-d), at increasing phage titers (10³, 10⁹ and 10¹⁰ pfu/ml). The effect of the engineered phages versus that of the empty M13 phage control was compared. The effect on fibrillar mass concentration was determined at the highest phage titer (10¹⁰ pfu/ml) for each AB-phage and M13 from the averaged end-point ThT emission (FIG. 2-e).

In an embodiment, a lag phase is observed before the formation of the first protofibrils (FIG. 2-a-d). The length of this lag time is dependent on the concentration of Aβ42 monomers that are available for fibril formation. Once the first protofibrils are formed, they serve as catalysts for the generation of additional fibrils, which is reflected by the rapid increase in ThT fluorescence. The plateau value is indicative for the total amount of end-stage Aβ42 fibrils formed.

In an embodiment, addition of M13 phages (i.e., those without a displayed peptide) had a discrete effect on the lag phase (FIG. 2-a) on the aggregation half time (t_{1/2}), which is defined as the time point when the ThT intensity reaches half way between the initial baseline and the final plateau value, but they did decrease the levels of end-point fibrils at increasing titers (FIGS. 2-e). This might be due to unspecific interactions between M13 coat proteins and Aβ42 monomers.

In an embodiment, the tested engineered phages had distinct effects on Aβ42 aggregation. The Aβ30-39 phages inhibited Aβ42 aggregation and kinetics and results in a lower fibril mass/ThT intensity at the end point (FIG. 2-c). At increasing titers, Aβ30-39 progressively delayed Aβ42 aggregation as noted by an increase of ˜1 h in the half time (Table 2). Simultaneously, end point fibril mass was also substantially reduced (FIG. 2-e). On the other hand, Aβ36-39 and Aβ33-42 did not show a significative effect over Aβ fibril mass nor on Aβ42 aggregation rate, in comparison to M13 bacteriophage controls (FIG. 2-b, 2-d), even at the highest tested titer.

In an embodiment, the segment AB30-39 interacts more effectively with AI monomers and also with a broader ensemble of early Aβ polymorphs, as showed by the stronger suppression of fibril formation observed.

In an embodiment, the effect of phage-displayed peptides on secondary nucleation of monomers on the surfaces of amyloid fibrils was determined. The aggregation of Aβ comprises several microscopic steps that include primary nucleation of Aβ monomers, secondary nucleation of monomers on the surface of fibrils and elongation of fibrils resulting from the addition of monomers (FIG. 3-a): primary nucleation (kₙ) starting from monomers, elongation (k₊) by monomer addiction to existing aggregates, and secondary nucleation (k₂) from nucleation of monomers on the fibril surface¹⁸.

In an embodiment, seeded experiments in which pre-formed Aβ42 fibrils (2%) are added to monomeric Aβ42 allow to assess the contribution of secondary nucleation, as pre-formed fibrils provide reactive surfaces. These assays allow to identify inhibitors that block secondary nucleation of Aβ42 monomers on the surfaces of amyloid fibrils. In an embodiment, pre-formed fibrils were added to Aβ42 monomers, resulting in an acceleration of fibril formation independent of primary nucleation. Phages were added to this reaction at a high concentration (10¹⁰ pfu/ml). The results obtained showed that under the tested experimental conditions, the seeded aggregation of Aβ42 (t_{1/2}=1.09±0.01 h) proceeds twice faster than the unseeded one (t_{1/2}=2.34±0.1 h) (FIG. 3b-f).

In an embodiment, the effect of the empty M13 bacteriophage has a negligible effect on the rate of seeded reaction, while AB30-39 seems to be an efficient inhibitor of secondary nucleation of monomers on the surface of fibrils as the reaction is slowed down (t_{1/2}=2.69±0.22 h). Interestingly, a distinct situation is observed for Aβ33-42, which causes a slight enhancement in the seeded aggregation rate (t_{1/2}=0.63±0.04 h) in respect to that observed for the M13 control (t_{1/2}=1.01±0.05 h) (FIG. 3-f). This implies that the Aβ33-42 motif is itself capable of seeding Aβ42 monomers, which can be explained by the context of the structural characteristics of Aβ42 amyloid fibrils¹⁹. Indeed, it has been pointed out that the hydrophobic strip formed by residues Val40 and Ala42 that runs down the outer surface of the protofilament could enhance secondary nucleation. These are precisely residues which are present in AB33-42 but not in AB30-39, thus providing a possible explanation for the results obtained.

In and embodiment, the effect of engineered phages on fibril content at the end-point of the aggregation curves (at 10¹⁰ pfu/ml) was qualitatively assessed in immunoblots using the anti-amyloid fibril OC antibody and phages at a high concentration (10¹⁰ pfu/ml) (FIG. 4). A decreased amount of fibrillar Aβ was observed in the assays in the presence of both engineered phages AB30-39 and AB33-42 but not AB36-30 phage (FIG. 4).

In and embodiment, both AB30-39 and AB33-42 phages effectively influence the formation of Aβ42 fibrils departing from monomeric Aβ42, but not AB36-39 thus not being effective for the intended purpose.

In an embodiment, the ability of the AB-phages to recognize Aβ aggregates in hippocampal slices of APP/PS1 transgenic mice was determined. It was examined whether the engineered phages are capable of detecting natural A-aggregates in brain tissue from Aβ42-overproducing mice. Mice that express human APP and mutant presenilin 1 were used as models for early-onset AD²⁰. These APP/PS1-transgenic mice start showing spine loss and altered network activity in hippocampus accompanied with hippocampus-dependent memory impairment as early as 3-4 months of age²⁰. However, amyloid plaques can only be detected in the hippocampus of these mice when they are ≥6 months of age, suggesting Aβ-oligomers are influencing neuronal function well before plaques are formed. Brains of 3-4 months old and 9-10 months old APP/PS1-transgenic and wild-type mice were isolated and immunohistochemistry was performed on brain slices (FIG. 5). An immunostaining with an anti-Aβ antibody (6E10) visualized plaques in the CA1 region of 9-10 months old APP/PS1-mice, but not in those aged 3-4 months (FIG. 5A-d4).

FIG. 5A shows representative fluorescence images of control and APP mice brain tissue samples in two different age groups: 3-4 months and 9-12 months, and signal quantification. Samples were incubated with 10⁸ pfu/ml of M13 (FIG. 5A-a), AB30-39 (FIG. 5A-b) and AB33-42 phages (FIG. 5A-c). The anti-fd phage was diluted 1:1000 and the 6E10 (1:1000 diluted) antibody staining was also performed for the identification of Aβ plaques (FIG. 5A-d). Phages are represented in green, the Aβ species in red and the cellular nuclei in blue. The signal quantification was performed for each individual phage in WT and APP mice samples for both age groups (FIGS. 5Be and f) and in the cell body region and stratum radiatum (FIGS. 5B-g, h and 5C-i, j). The results plotted show means±SE of n=4 samples. Statistical comparisons were performed using Students t-test, ** when p<0.001 and * when p<0.05.

The brain slices were exposed to the phages and subsequently stained with an anti-M13 mAb. M13 phages without Aβ-peptide motif did not show any staining in either WT or APP/PS1 mice (FIG. 5A-a). In contrast, both AB30-39 and AB33-42 phages showed a puncta staining in CA1 of brain samples of APP/PS1-mice (FIGS. 5A-b2, b4, c2, c4). These puncta are substantially smaller in size than amyloid plaques, and the density of these puncta significantly increased with age (FIG. 5B-f). In 3-4 months' old mice, these puncta are predominantly present in the cell body region, with lower levels in stratum radiatum, while in 9-12 months old mice the puncta were evenly distributed among CA1 cell body and dendritic areas (FIGS. 5B-g, h). AB33-42 phages showed a consistently higher number of puncta in the CA1 region of APP/PS1-mice when compared with AB30-39 (FIGS. 5B-e, f). This observation is in line with a previous analysis of binding affinities of Aβ30-39 and Aβ33-42 peptides for Aβ fibrils and oligomers: Aβ30-39 is only capable of recognizing fibrils and oligomers above 36 ng while the Aβ33-42 peptide sequence recognizes fibrils above 2.4 ng and oligomers above 5.8 ng¹¹. In comparison to APP/PS1-mice, staining of AB30-39 and AB33-42 is largely absent in brain tissue from WT mice (FIG. 5A-b1, A-b3, A-c1, A-c3 or 4B-e, f). Low level of staining by AB30-39 and AB33-42 phages was detected in the elderly group of WT animals (FIGS. 5A-b3, c3), which corresponds with the existence of oligomeric Aβ in aging WT mice²⁶. These data show that the phages displaying Aβ peptides can be used as an immunohistochemistry tool to detect small Aβ aggregates in brain slices of mice.

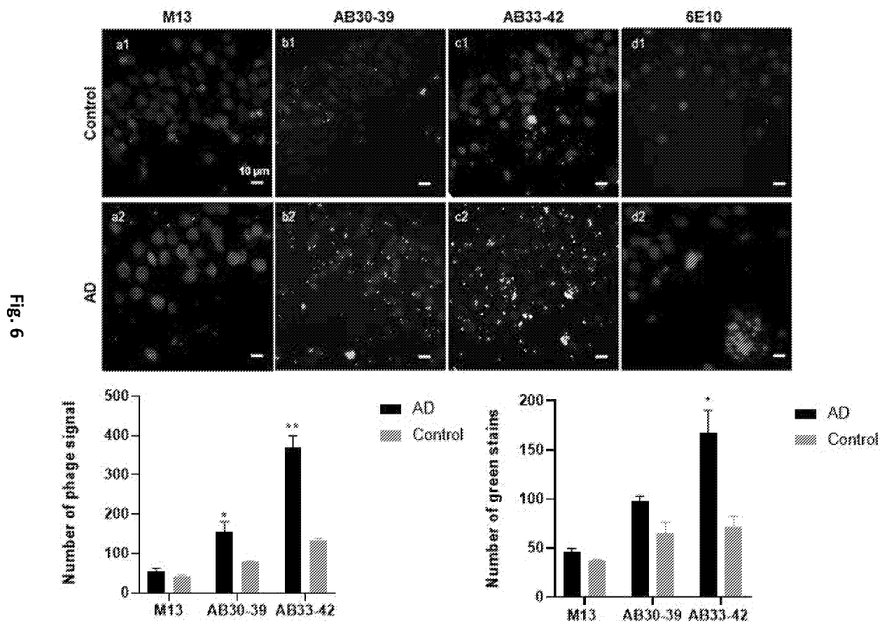

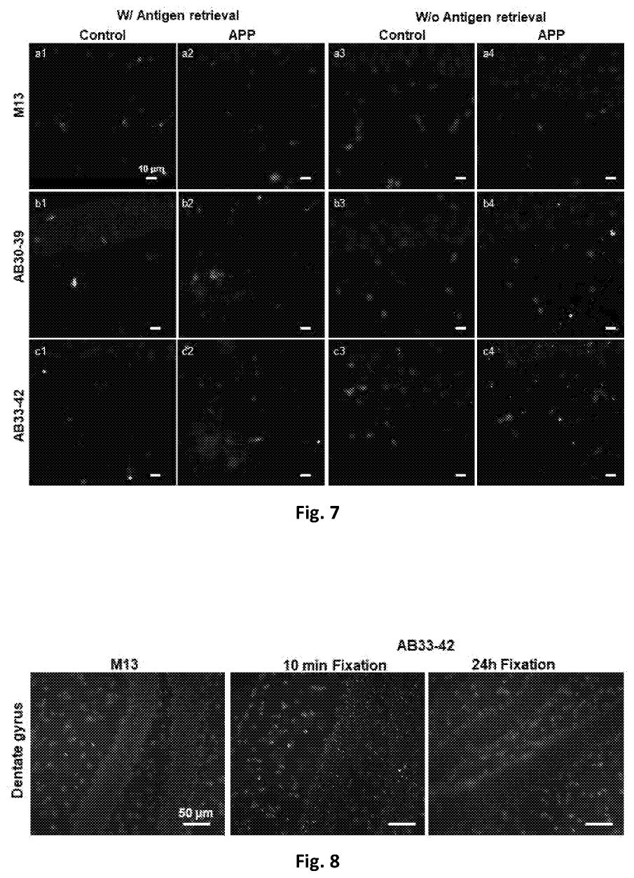

AB30-39 and AB33-42 phages only stained small (<1 μm) species of Aβ aggregates in APP/PS1-mice, and failed to detect amyloid plaques. A direct analysis of AB30-39 or AB33-42 phage versus 6E10 staining was not possible because antigen retrieval which involves thermal denaturation of secondary and tertiary protein structures and is necessary for primary antibody 6E10 to detect plaques prevented the binding of AB30-39 and AB33-42 phages to brain tissue of APP/PS1-mice (FIG. 7). AB33-42 staining proved also unsuccessful when brain samples were fixated with paraformaldehyde for extended periods of time (FIG. 8). These observations suggest that Aβ oligomers and (proto)fibrils in tissue need to be in a natural configuration to be recognized by AB30-39 or AB33-42 phages.

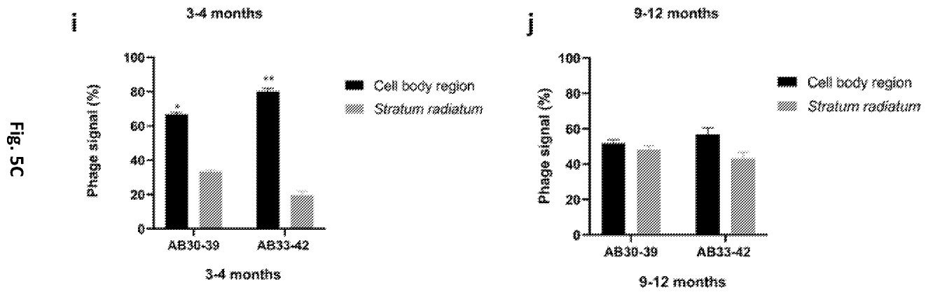

In an embodiment, the ability of AB-phages to recognize Aβ aggregates in hippocampus of AD-patients were determined. It was assessed whether AB30-39 and AB33-42 phages were capable of detecting Aβ aggregates in human brain samples. Cryopreserved brain samples of the hippocampus from three AD-patients and three age-matched healthy controls (Table 1) were stained with AB30-39, AB33-42 and control M13 phages (FIG. 6).

FIG. 6 shows representative fluorescence images of control and AD human brain tissue samples and signal quantification. Samples were incubated with 10⁸ pfu/ml of M13 (FIG. 6-a), AB30-39 (FIG. 6-b) and AB33-42 (FIG. 6-c) phages. The anti-fd phage was diluted 1:1000 and the 6E10 (1:1000 diluted) antibody staining was also performed for the identification of Aβ plaques (FIG. 6-d). Phages are represented in green, the Aβ species in red and the cellular nuclei in blue. For each individual phage in control and AD human samples the signal quantification (FIG. 6-e) and the green puncta quantification (FIG. 6-f) was performed. The results plotted show means±SE of n=3 samples. Statistical comparisons were performed using Students t-test, ** when p<0.001 and * when p<0.05.

Both AB30-39 and AB33-42 phages, but not control phages, showed substantial staining in AD-samples that was significantly higher than their staining in hippocampal samples from control individuals (p<0.05 and p<0.001, respectively). Similarly, it was observed in mouse samples that AB33-42 phages gave enhanced staining in AD-samples compared with AB30-39 phages. Both AB30-39 and AB33-42 phages detected, in addition to the small aggregates that were also observed in APP/PS1-mice, also slightly larger aggregates (˜10 μm), but did not detect those that are the size of plaques, as identified with 6E10 staining in these human samples (FIG. 6-d2). Although the existence of Aβ plaques in cognitively healthy individuals is not uncommon at an advanced age²¹, 6E10 staining showed only minimal amounts of plaques in brain samples from the three age-matched control individuals, which corresponds with a lower level of AB30-39 and AB33-42 staining. These data indicate that AB30-39 and AB33-42 phages can be used to detect small Aβ aggregates in human post-mortem brain tissue.

In an embodiment, the ability of AD-derived peptides displayed on M13 phages to interact with Aβ-aggregates was determined. 10 aa-long peptides corresponding with the carboxy-terminal region of AD that were previously shown to have low (μM) affinity for Aβ monomers and high (nM) affinity for Aβ oligomers and fibrils¹¹ were used. Because each phage presents 5 to 8 peptides at its surface, AB30-39 and AB33-42 phages bind Aβ-aggregates with higher avidity and likely also with higher specificity for oligomers in relation to monomers than individual Aβ3-peptides. The Aβ-specific phages were tested for their ability to prevent and/or inhibit Aβ aggregation and while M13 phages by themselves already possess a slight capacity to destabilize Aβ fibrils as previously reported²², AB30-39 show a superior capacity to do so, primarily through the inhibition of secondary nucleation of Aβ42 monomers on the surfaces of amyloid fibrils.

In an embodiment, the ability of both AB30-39 and AB33-42 phages to recognize natural aggregates of Aβ was also determined. In brain tissue of APP/PS1-transgenic mice, both phages detect aggregates of Aβ that are substantially smaller than amyloid plaques. These phages are capable of detecting Aβ oligomers and fibrils by immunofluorescence in brain slices, and as they practically are inexpensive and easy to produce, these phages could provide a good alternative in comparison to commercially available antibodies. Aβ33-42 phages are able to detect a larger amount of Aβ aggregates than AB30-39, both in mouse and human AD-brain samples, which is likely due to the higher affinity of the Aβ33-42 peptide for Aβ-oligomers. It was observed that these phages only detect Aβ-oligomers provided that secondary protein structures in the brain tissue are kept largely intact, as exposure to paraformaldehyde for extended time periods or antigen retrieval procedures substantially obscure this phage staining of Aβ-aggregates.

In an embodiment, AB33-42 phages were used as an immunohistochemistry tool to study Aβ aggregates in postmortem brain tissue. It was observed that Aβ aggregates are present in the CA1 region of the hippocampus in 3-months old APP/PS1-mice, which are likely responsible for the synaptic memory deficits these mice can experience at this age. It was further observed that surprisingly, at this early age, Aβ-oligomers are predominantly found in the stratum pyramidal in comparison to the stratum radiatum.

### Examples

As an example, for use as a diagnostic tool in immunoassays, the engineered phages at a concentration of 10⁴ to 10¹⁰ pfu/ml are stored at 4° C. in a buffer solution of TBS 1× or PBS 1×. The phages are incubated with tissue samples at a concentration of 10⁴-10¹⁰ pfu/ml (if necessary, dilute the phages in TBST 1×). The amount of phage to be add to the tissue varies between 50-100 μl. The reaction takes place at 4° C. in a humidified chamber overnight.

As an example, of method of diagnostic using the engineered phages, tissue slides were washed in TBST five times for 10 minutes each time, and subsequently incubated overnight at 4° C. in a humidified chamber with 1:1000 diluted rabbit anti-fd phage antibody. It was then washed and incubated for 2 hours at room temperature with 1:200 diluted FITC-labeled goat anti-rabbit IgG antibody. Tissue slides were covered with Vectashield mounting medium with DAPI and images acquired with a fluorescence microscope.

### Example of Prevention

As an example, for use as a preventive tool, the engineered phage AB30-39 is administered in a saline solution (PBS 1×) at a concentration between 10⁴ to 10¹⁰ pfu/ml. No more than 20-100 ml/day can be administered. Multiple doses may be necessary during a given period of time. Cognitive and behavioral tests are performed to assess the phage capability to prevent spine loss and memory deficits.

### Example of Treatment

As an example, for use as a therapeutic tool, the engineered phage AB30-39 is administered in a saline solution (PBS 1×) at a concentration between 10⁴ to 10¹⁰ pfu/ml. No more than 20-100 ml/day can be administered. Multiple doses may be necessary during a given period of time. Cognitive and behavioral tests are performed to assess the phage capability to prevent spine loss and memory deficits.

In an embodiment, it was observed that AB30-39 phages have the capacity to prevent the aggregation of Aβ42 in vitro.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above-described embodiments are combinable.

The following dependent claims further set out particular embodiments of the disclosure.

SEQUENCE LIST:

| SEQ | | | | | |
|---|---|---|---|---|---|
| ID | Identi- | | Molecule | | |
| NO. | fication | Organism | type | Type | Sequence |
| 1 | Aβ30-39 | Synthetic | Aminoacid | Protein | AIIGLMVGGV |
| 2 | Aβ33-42 | Synthetic | Aminoacid | Protein | GLMVGGVVIA |
| 3 | pETDuet-1 | Synthetic | DNA | Other DNA | ggggaattgtgagcggataacaattcccctctagaaataattttgtttaactttaagaaggagatataccatgggcagcagccatcacc |
| | | | | | atcatcaccacagccaggatccgaattcgagctcggcgcgcctgcaggtcgacaagcttgcggccgcataatgcttaagtcgaacaga |
| | | | | | aagtaatcgtattgtacacggccgcataatcgaaattaatacgactcactataggggaattgtgagcggataacaattccccatcttag |
| | | | | | tatattagttaagtataagaaggagatatacatatggcagatctcaattggatatcggccggccacgcgatcgctgacgtcggtaccct |
| | | | | | cgagtctggtaaagaaaccgctgctgcgaaatttgaacgccagcacatggactcgtctactagcgcagcttaattaacctaggctgctg |
| | | | | | ccaccgctgagcaataactagcataaccccttggggcctctaaacgggtcttgaggggttttttgctgaaaggaggaactatatccgga |
| | | | | | ttggcgaatgggacgcgccctgtagcggcgcattaagcgcggcgggtgtggtggttacgcgcagcgtgaccgctacacttgccagcgc |
| | | | | | cctagcgcccgctcctttcgctttcttcccttcctttctcgccacgttcgccggctttccccgtcaagctctaaatcgggggctccctttagg |
| | | | | | gttccgatttagtgctttacggcacctcgaccccaaaaaacttgattagggtgatggttcacgtagtgggccatcgccctgatagacggt |
| | | | | | ttttcgccctttgacgttggagtccacgttctttaatagtggactcttgttccaaactggaacaacactcaaccctatctcggtctattcttt |
| | | | | | tgatttataagggattttgccgatttcggcctattggttaaaaaatgagctgatttaacaaaaatttaacgcgaattttaacaaaatatta |
| | | | | | acgtttacaatttctggcggcacgatggcatgagattatcaaaaaggatcttcacctagatccttttaaattaaaaatgaagttttaaat |
| | | | | | caatctaaagtatatatgagtaaacttggtctgacagttaccaatgcttaatcagtgaggcacctatctcagcgatctgtctatttcgttc |
| | | | | | atccatagttgcctgactccccgtcgtgtagataactacgatacgggagggcttaccatctggccccagtgctgcaatgataccgcgag |
| | | | | | acccacgctcaccggctccagatttatcagcaataaaccagccagccggaagggccgagcgcagaagtggtcctgcaactttatccgc |
| | | | | | ctccatccagtctattaattgttgccgggaagctagagtaagtagttcgccagttaatagtttgcgcaacgttgttgccattgctacaggc |
| | | | | | atcgtggtgtcacgctcgtcgtttggtatggcttcattcagctccggttcccaacgatcaaggcgagttacatgatcccccatgttgtgca |
| | | | | | aaaaagcggttagctccttcggtcctccgatcgttgtcagaagtaagttggccgcagtgttatcactcatggttatggcagcactgcata |
| | | | | | attctcttactgtcatgccatccgtaagatgcttttctgtgactggtgagtactcaaccaagtcattctgagaatagtgtatgcggcgacc |
| | | | | | gagttgctcttgcccggcgtcaatacgggataataccgcgccacatagcagaactttaaaagtgctcatcattggaaaacgttcttcgg |
| | | | | | ggcgaaaactctcaaggatcttaccgctgttgagatccagttcgatgtaacccactcgtgcacccaactgatcttcagcatcttttacttt |
| | | | | | caccagcgtttctgggtgagcaaaaacaggaaggcaaaatgccgcaaaaaagggaataagggcgacacggaaatgttgaatactc |
| | | | | | atactcttcctttttcaatcatgattgaagcatttatcagggttattgtctcatgagcggatacatatttgaatgtatttagaaaaataaac |
| | | | | | aaataggtcatgaccaaaatcccttaacgtgagttttcgttccactgagcgtcagaccccgtagaaaagatcaaaggatcttcttgaga |
| | | | | | tcctttttttctgcgcgtaatctgctgcttgcaaacaaaaaaaccaccgctaccagcggtggtttgtttgccggatcaagagctaccaact |
| | | | | | ctttttccgaaggtaactggcttcagcagagcgcagataccaaatactgtccttctagtgtagccgtagttaggccaccacttcaagaac |
| | | | | | tctgtagcaccgcctacatacctcgctctgctaatcctgttaccagtggctgctgccagtggcgataagtcgtgtcttaccgggttggact |
| | | | | | caagacgatagttaccggataaggcgcagcggtcgggctgaacggggggttcgtgcacacagcccagcttggagcgaacgacctac |
| | | | | | accgaactgagatacctacagcgtgagctatgagaaagcgccacgcttcccgaagggagaaaggcggacaggtatccggtaagcgg |
| | | | | | cagggtcggaacaggagagcgcacgagggagcttccagggggaaacgcctggtatctttatagtcctgtcgggtttcgccacctctga |
| | | | | | cttgagcgtcgatttttgtgatgctcgtcaggggggcggagcctatggaaaaacgccagcaacgcggcctttttacggttcctggccttt |
| | | | | | tgctggccttttgctcacatgttctttcctgcgttatcccctgattctgtggataaccgtattaccgcctttgagtgagctgataccgctcgc |
| | | | | | cgcagccgaacgaccgagcgcagcgagtcagtgagcgaggaagcggaagagcgcctgatgcggtattttctccttacgcatctgtgc |
| | | | | | ggtatttcacaccgcatatatggtgcactctcagtacaatctgctctgatgccgcatagttaagccagtatacactccgctatcgctacgt |
| | | | | | gactgggtcatggctgcgccccgacacccgccaacacccgctgacgcgccctgacgggcttgtctgctcccggcatccgcttacagac |
| | | | | | aagctgtgaccgtctccgggagctgcatgtgtcagaggttttcaccgtcatcaccgaaacgcgcgaggcagctgcggtaaagctcatc |
| | | | | | agcgtggtcgtgaagcgattcacagatgtctgcctgttcatccgcgtccagctcgttgagtttctccagaagcgttaatgtctggcttctg |
| | | | | | ataaagcgggccatgttaagggcggttttttcctgtttggtcactgatgcctccgtgtaagggggatttctgttcatgggggtaatgatac |
| | | | | | cgatgaaacgagagaggatgctcacgatacgggttactgatgatgaacatgcccggttactggaacgttgtgagggtaaacaactgg |
| | | | | | cggtatggatgcggcgggaccagagaaaaatcactcagggtcaatgccagcgcttcgttaatacagatgtaggtgttccacagggtag |
| | | | | | ccagcagcatcctgcgatgcagatccggaacataatggtgcagggcgctgacttccgcgtttccagactttacgaaacacggaaaccg |
| | | | | | aagaccattcatgttgttgctcaggtcgcagacgttttgcagcagcagtcgcttcacgttcgctcgcgtatcggtgattcattctgctaac |
| | | | | | cagtaaggcaaccccgccagcctagccgggtcctcaacgacaggagcacgatcatgctagtcatgccccgcgcccaccggaaggag |
| | | | | | ctgactgggttgaaggctctcaagggcatcggtcgagatcccggtgcctaatgagtgagctaacttacattaattgcgttgcgctcactg |
| | | | | | cccgctttccagtcgggaaacctgtcgtgccagctgcattaatgaatcggccaacgcgcggggagaggcggtttgcgtattgggcgcc |
| | | | | | agggtggtttttcttttcaccagtgagacgggcaacagctgattgcccttcaccgcctggccctgagagagttgcagcaagcggtccac |
| | | | | | gctggtttgccccagcaggcgaaaatcctgtttgatggtggttaacggcgggatataacatgagctgtcttcggtatcgtcgtatcccac |
| | | | | | taccgagatgtccgcaccaacgcgcagcccggactcggtaatggcgcgcattgcgcccagcgccatctgatcgttggcaaccagcatc |
| | | | | | gcagtgggaacgatgccctcattcagcatttgcatggtttgttgaaaaccggacatggcactccagtcgccttcccgttccgctatcggc |
| | | | | | tgaatttgattgcgagtgagatatttatgccagccagccagacgcagacgcgccgagacagaacttaatgggcccgctaacagcgcg |
| | | | | | atttgctggtgacccaatgcgaccagatgctccacgcccagtcgcgtaccgtcttcatgggagaaaataatactgttgatgggtgtctg |
| | | | | | gtcagagacatcaagaaataacgccggaacattagtgcaggcagcttccacagcaatggcatcctggtcatccagcggatagttaat |
| | | | | | gatcagcccactgacgcgttgcgcgagaagattgtgcaccgccgctttacaggcttcgacgccgcttcgttctaccatcgacaccacca |
| | | | | | cgctggcacccagttgatcggcgcgagatttaatcgccgcgacaatttgcgacggcgcgtgcagggccagactggaggtggcaacgc |
| | | | | | caatcagcaacgactgtttgcccgccagttgttgtgccacgcggttgggaatgtaattcagctccgccatcgccgcttccactttttcccg |
| | | | | | cgttttcgcagaaacgtggctggcctggttcaccacgcgggaaacggtctgataagagacaccggcatactctgcgacatcgtataac |
| | | | | | gttactggtttcacattcaccaccctgaattgactctcttccgggcgctatcatgccataccgcgaaaggttttgcgccattcgatggtgt |
| | | | | | ccgggatctcgacgctctcccttatgcgactcctgcattaggaagcagcccagtagtaggttgaggccgttgagcaccgccgccgcaa |
| | | | | | ggaatggtgcatgcaaggagatggcgcccaacagtcccccggccacggggcctgccaccatacccacgccgaaacaagcgctcatg |
| | | | | | agcccgaagtggcgagcccgatcttccccatcggtgatgtcggcgatataggcgccagcaaccgcacctgtggcgccggtgatgccgg |
| | | | | | ccacgatgcgtccggcgtagaggatcgagatcgatctcgatcccgcgaaattaatacgactcactata |
| 4 | pETDuet-1 | Synthetic | DNA | Other DNA | ggggaattgtgagcggataacaattcccctctagaaataattttgtttaactttaagaaggagatataccatgggcagcagccatcacc |
| | with gene | | | | atcatcaccacagccaggatccatgaaatacctattgcctacggcagccgctggattgttattactcgcggcccagccggccatggccg |
| | pelB | | | | aattcgagctcggcgcgcctgcaggtcgacaagcttgtgaaaaaattattattcgcaattcctttagtggtacctttctattctcactcgg |
| | and gene | | | | ccgaaactgttgaaagttgtttagcaaaatcccatacagaaaattcatttactaacgtctggaaagacgacaaaactttagatcgttac |
| | 3 of | | | | gctaactatgagggctgtctgtggaatgctacaggcgttgtagtttgtactggtgacgaaactcagtgttacggtacatgggttcctatt |
| | M13 phage | | | | gggcttgctatccctgaaaatgagggtggtggctctgagggtggcggttctgagggtggcggttctgagggtggcggtactaaacctcc |
| | (inter- | | | | tgagtacggtgatacacctattccgggctatacttatatcaaccctctcgacggcacttatccgcctggtactgagcaaaaccccgctaa |
| | mediate | | | | tcctaatccttctcttgaggagtctcagcctcttaatactttcatgtttcagaataataggttccgaaataggcagggggcattaactgttt |
| | plasmid) | | | | atacgggcactgttactcaaggcactgaccccgttaaaacttattaccagtacactcctgtatcatcaaaagccatgtatgacgcttact |
| | | | | | ggaacggtaaattcagagactgcgctttccattctggctttaatgaggatttatttgtttgtgaatatcaaggccaatcgtctgacctgcc |
| | | | | | tcaacctcctgtcaatgctggcggcggctctggtggtggttctggtggcggctctgagggtggtggctctgagggtggcggttctgaggg |
| | | | | | tggcggctctgagggaggcggttccggtggtggctctggttccggtgattttgattatgaaaagatggcaaacgctaataagggggcta |
| | | | | | tgaccgaaaatgccgatgaaaacgcgctacagtctgacgctaaaggcaaacttgattctgtcgctactgattacggtgctgctatcgat |
| | | | | | ggtttcattggtgacgtttccggccttgctaatggtaatggtgctactggtgattttgctggctctaattcccaaatggctcaagtcggtga |
| | | | | | cggtgataattcacctttaatgaataatttccgtcaatatttaccttccctccctcaatcggttgaatgtcgcccttttgtctttggcgctgg |
| | | | | | taaaccatatgaattttctattgattgtgacaaaataaacttattccgtggtgtctttgcgtttcttttatatgttgccacctttatgtatgta |
| | | | | | ttttctacgtttgctaacatactgcgtaataaggagtcttaagcggccgcataatgcttaagtcgaacagaaagtaatcgtattgtacac |
| | | | | | ggccgcataatcgaaattaatacgactcactataggggaattgtgagcggataacaattccccatcttagtatattagttaagtataag |
| | | | | | aaggagatatacatatggcagatctcaattggatatcggccggccacgcgatcgctgacgtcggtaccctcgagtctggtaaagaaac |
| | | | | | cgctgctgcgaaatttgaacgccagcacatggactcgtctactagcgcagcttaattaacctaggctgctgccaccgctgagcaataac |
| | | | | | tagcataaccccttggggcctctaaacgggtcttgaggggttttttgctgaaaggaggaactatatccggattggcgaatgggacgcgc |
| | | | | | cctgtagcggcgcattaagcgcggcgggtgtggtggttacgcgcagcgtgaccgctacacttgccagcgccctagcgcccgctcctttc |
| | | | | | gctttcttcccttcctttctcgccacgttcgccggctttccccgtcaagctctaaatcgggggctccctttagggttccgatttagtgcttta |
| | | | | | cggcacctcgaccccaaaaaacttgattagggtgatggttcacgtagtgggccatcgccctgatagacggtttttcgccctttgacgttg |
| | | | | | gagtccacgttctttaatagtggactcttgttccaaactggaacaacactcaaccctatctcggtctattcttttgatttataagggatttt |
| | | | | | gccgatttcggcctattggttaaaaaatgagctgatttaacaaaaatttaacgcgaattttaacaaaatattaacgtttacaatttctgg |
| | | | | | cggcacgatggcatgagattatcaaaaaggatcttcacctagatccttttaaattaaaaatgaagttttaaatcaatctaaagtatatat |
| | | | | | gagtaaacttggtctgacagttaccaatgcttaatcagtgaggcacctatctcagcgatctgtctatttcgttcatccatagttgcctgac |
| | | | | | tccccgtcgtgtagataactacgatacgggagggcttaccatctggccccagtgctgcaatgataccgcgagacccacgctcaccggct |
| | | | | | ccagatttatcagcaataaaccagccagccggaagggccgagcgcagaagtggtcctgcaactttatccgcctccatccagtctatta |
| | | | | | attgttgccgggaagctagagtaagtagttcgccagttaatagtttgcgcaacgttgttgccattgctacaggcatcgtggtgtcacgct |
| | | | | | cgtcgtttggtatggcttcattcagctccggttcccaacgatcaaggcgagttacatgatcccccatgttgtgcaaaaaagcggttagct |
| | | | | | ccttcggtcctccgatcgttgtcagaagtaagttggccgcagtgttatcactcatggttatggcagcactgcataattctcttactgtcatg |
| | | | | | ccatccgtaagatgcttttctgtgactggtgagtactcaaccaagtcattctgagaatagtgtatgcggcgaccgagttgctcttgcccg |
| | | | | | gcgtcaatacgggataataccgcgccacatagcagaactttaaaagtgctcatcattggaaaacgttcttcggggcgaaaactctcaa |
| | | | | | ggatcttaccgctgttgagatccagttcgatgtaacccactcgtgcacccaactgatcttcagcatcttttactttcaccagcgtttctggg |
| | | | | | tgagcaaaaacaggaaggcaaaatgccgcaaaaaagggaataagggcgacacggaaatgttgaatactcatactcttcctttttcaa |
| | | | | | tcatgattgaagcatttatcagggttattgtctcatgagcggatacatatttgaatgtatttagaaaaataaacaaataggtcatgacca |
| | | | | | aaatcccttaacgtgagttttcgttccactgagcgtcagaccccgtagaaaagatcaaaggatcttcttgagatcctttttttctgcgcgt |
| | | | | | aatctgctgcttgcaaacaaaaaaaccaccgctaccagcggtggtttgtttgccggatcaagagctaccaactctttttccgaaggtaa |
| | | | | | ctggcttcagcagagcgcagataccaaatactgtccttctagtgtagccgtagttaggccaccacttcaagaactctgtagcaccgcct |
| | | | | | acatacctcgctctgctaatcctgttaccagtggctgctgccagtggcgataagtcgtgtcttaccgggttggactcaagacgatagtta |
| | | | | | ccggataaggcgcagcggtcgggctgaacggggggttcgtgcacacagcccagcttggagcgaacgacctacaccgaactgagata |
| | | | | | cctacagcgtgagctatgagaaagcgccacgcttcccgaagggagaaaggcggacaggtatccggtaagcggcagggtcggaacag |
| | | | | | gagagcgcacgagggagcttccagggggaaacgcctggtatctttatagtcctgtcgggtttcgccacctctgacttgagcgtcgatttt |
| | | | | | tgtgatgctcgtcaggggggcggagcctatggaaaaacgccagcaacgcggcctttttacggttcctggccttttgctggccttttgctc |
| | | | | | acatgttctttcctgcgttatcccctgattctgtggataaccgtattaccgcctttgagtgagctgataccgctcgccgcagccgaacgac |
| | | | | | cgagcgcagcgagtcagtgagcgaggaagcggaagagcgcctgatgcggtattttctccttacgcatctgtgcggtatttcacaccgc |
| | | | | | atatatggtgcactctcagtacaatctgctctgatgccgcatagttaagccagtatacactccgctatcgctacgtgactgggtcatggc |
| | | | | | tgcgccccgacacccgccaacacccgctgacgcgccctgacgggcttgtctgctcccggcatccgcttacagacaagctgtgaccgtct |
| | | | | | ccgggagctgcatgtgtcagaggttttcaccgtcatcaccgaaacgcgcgaggcagctgcggtaaagctcatcagcgtggtcgtgaag |
| | | | | | cgattcacagatgtctgcctgttcatccgcgtccagctcgttgagtttctccagaagcgttaatgtctggcttctgataaagcgggccatg |
| | | | | | ttaagggcggttttttcctgtttggtcactgatgcctccgtgtaagggggatttctgttcatgggggtaatgataccgatgaaacgagag |
| | | | | | aggatgctcacgatacgggttactgatgatgaacatgcccggttactggaacgttgtgagggtaaacaactggcggtatggatgcggc |
| | | | | | gggaccagagaaaaatcactcagggtcaatgccagcgcttcgttaatacagatgtaggtgttccacagggtagccagcagcatcctgc |
| | | | | | gatgcagatccggaacataatggtgcagggcgctgacttccgcgtttccagactttacgaaacacggaaaccgaagaccattcatgtt |
| | | | | | gttgctcaggtcgcagacgttttgcagcagcagtcgcttcacgttcgctcgcgtatcggtgattcattctgctaaccagtaaggcaaccc |
| | | | | | cgccagcctagccgggtcctcaacgacaggagcacgatcatgctagtcatgccccgcgcccaccggaaggagctgactgggttgaag |
| | | | | | gctctcaagggcatcggtcgagatcccggtgcctaatgagtgagctaacttacattaattgcgttgcgctcactgcccgctttccagtcg |
| | | | | | ggaaacctgtcgtgccagctgcattaatgaatcggccaacgcgcggggagaggcggtttgcgtattgggcgccagggtggtttttcttt |
| | | | | | tcaccagtgagacgggcaacagctgattgcccttcaccgcctggccctgagagagttgcagcaagcggtccacgctggtttgccccag |
| | | | | | caggcgaaaatcctgtttgatggtggttaacggcgggatataacatgagctgtcttcggtatcgtcgtatcccactaccgagatgtccgc |
| | | | | | accaacgcgcagcccggactcggtaatggcgcgcattgcgcccagcgccatctgatcgttggcaaccagcatcgcagtgggaacgat |
| | | | | | gccctcattcagcatttgcatggtttgttgaaaaccggacatggcactccagtcgccttcccgttccgctatcggctgaatttgattgcga |
| | | | | | gtgagatatttatgccagccagccagacgcagacgcgccgagacagaacttaatgggcccgctaacagcgcgatttgctggtgaccc |
| | | | | | aatgcgaccagatgctccacgcccagtcgcgtaccgtcttcatgggagaaaataatactgttgatgggtgtctggtcagagacatcaa |
| | | | | | gaaataacgccggaacattagtgcaggcagcttccacagcaatggcatcctggtcatccagcggatagttaatgatcagcccactgac |
| | | | | | gcgttgcgcgagaagattgtgcaccgccgctttacaggcttcgacgccgcttcgttctaccatcgacaccaccacgctggcacccagtt |
| | | | | | gatcggcgcgagatttaatcgccgcgacaatttgcgacggcgcgtgcagggccagactggaggtggcaacgccaatcagcaacgact |
| | | | | | gtttgcccgccagttgttgtgccacgcggttgggaatgtaattcagctccgccatcgccgcttccactttttcccgcgttttcgcagaaac |
| | | | | | gtggctggcctggttcaccacgcgggaaacggtctgataagagacaccggcatactctgcgacatcgtataacgttactggtttcacat |
| | | | | | tcaccaccctgaattgactctcttccgggcgctatcatgccataccgcgaaaggttttgcgccattcgatggtgtccgggatctcgacgc |
| | | | | | tctcccttatgcgactcctgcattaggaagcagcccagtagtaggttgaggccgttgagcaccgccgccgcaaggaatggtgcatgca |
| | | | | | aggagatggcgcccaacagtcccccggccacggggcctgccaccatacccacgccgaaacaagcgctcatgagcccgaagtggcga |
| | | | | | gcccgatcttccccatcggtgatgtcggcgatataggcgccagcaaccgcacctgtggcgccggtgatgccggccacgatgcgtccgg |
| | | | | | cgtagaggatcgagatcgatctcgatcccgcgaaattaatacgactcactata |
| 5 | pETDuet-1 | Synthetic | DNA | Other DNA | Ggggaattgtgagcggataacaattcccctctagaaataattttgtttaactttaagaaggagatataccatgggcagcagccatcac |
| | inter- | | | | catcatcaccacagccaggatccatgaaatacctattgcctacggcagccgctggattgttattactcgcggcccagccggccatggcc |
| | mediate | | | | gaattcgagctcgcgattattggcctgatggtgggcggcgtggtcgacaagcttgtgaaaaaattattattcgcaattcctttagtggta |
| | plasmid + | | | | cctttctattctcactcggccgaaactgttgaaagttgtttagcaaaatcccatacagaaaattcatttactaacgtctggaaagacgac |
| | Aβ | | | | aaaactttagatcgttacgctaactatgagggctgtctgtggaatgctacaggcgttgtagtttgtactggtgacgaaactcagtgttac |
| | peptide | | | | ggtacatgggttcctattgggcttgctatccctgaaaatgagggtggtggctctgagggtggcggttctgagggtggcggttctgagggt |
| | Aβ30-39 | | | | ggcggtactaaacctcctgagtacggtgatacacctattccgggctatacttatatcaaccctctcgacggcacttatccgcctggtact |
| | (phagemid | | | | gagcaaaaccccgctaatcctaatccttctcttgaggagtctcagcctcttaatactttcatgtttcagaataataggttccgaaataggc |
| | AB30-39) | | | | agggggcattaactgtttatacgggcactgttactcaaggcactgaccccgttaaaacttattaccagtacactcctgtatcatcaaaag |
| | | | | | ccatgtatgacgcttactggaacggtaaattcagagactgcgctttccattctggctttaatgaggatttatttgtttgtgaatatcaagg |
| | | | | | ccaatcgtctgacctgcctcaacctcctgtcaatgctggcggcggctctggtggtggttctggtggcggctctgagggtggtggctctga |
| | | | | | gggtggcggttctgagggtggcggctctgagggaggcggttccggtggtggctctggttccggtgattttgattatgaaaagatggcaa |
| | | | | | acgctaataagggggctatgaccgaaaatgccgatgaaaacgcgctacagtctgacgctaaaggcaaacttgattctgtcgctactga |
| | | | | | ttacggtgctgctatcgatggtttcattggtgacgtttccggccttgctaatggtaatggtgctactggtgattttgctggctctaattccca |
| | | | | | aatggctcaagtcggtgacggtgataattcacctttaatgaataatttccgtcaatatttaccttccctccctcaatcggttgaatgtcgc |
| | | | | | ccttttgtctttggcgctggtaaaccatatgaattttctattgattgtgacaaaataaacttattccgtggtgtctttgcgtttcttttatatg |
| | | | | | ttgccacctttatgtatgtattttctacgtttgctaacatactgcgtaataaggagtcttaagcggccgcataatgcttaagtcgaacaga |
| | | | | | aagtaatcgtattgtacacggccgcataatcgaaattaatacgactcactataggggaattgtgagcggataacaattccccatcttag |
| | | | | | tatattagttaagtataagaaggagatatacatatggcagatctcaattggatatcggccggccacgcgatcgctgacgtcggtaccct |
| | | | | | cgagtctggtaaagaaaccgctgctgcgaaatttgaacgccagcacatggactcgtctactagcgcagcttaattaacctaggctgctg |
| | | | | | ccaccgctgagcaataactagcataaccccttggggcctctaaacgggtcttgaggggttttttgctgaaaggaggaactatatccgga |
| | | | | | ttggcgaatgggacgcgccctgtagcggcgcattaagcgcggcgggtgtggtggttacgcgcagcgtgaccgctacacttgccagcgc |
| | | | | | cctagcgcccgctcctttcgctttcttcccttcctttctcgccacgttcgccggctttccccgtcaagctctaaatcgggggctccctttagg |
| | | | | | gttccgatttagtgctttacggcacctcgaccccaaaaaacttgattagggtgatggttcacgtagtgggccatcgccctgatagacggt |
| | | | | | ttttcgccctttgacgttggagtccacgttctttaatagtggactcttgttccaaactggaacaacactcaaccctatctcggtctattcttt |
| | | | | | tgatttataagggattttgccgatttcggcctattggttaaaaaatgagctgatttaacaaaaatttaacgcgaattttaacaaaatatta |
| | | | | | acgtttacaatttctggcggcacgatggcatgagattatcaaaaaggatcttcacctagatccttttaaattaaaaatgaagttttaaat |
| | | | | | caatctaaagtatatatgagtaaacttggtctgacagttaccaatgcttaatcagtgaggcacctatctcagcgatctgtctatttcgttc |
| | | | | | atccatagttgcctgactccccgtcgtgtagataactacgatacgggagggcttaccatctggccccagtgctgcaatgataccgcgag |
| | | | | | acccacgctcaccggctccagatttatcagcaataaaccagccagccggaagggccgagcgcagaagtggtcctgcaactttatccgc |
| | | | | | ctccatccagtctattaattgttgccgggaagctagagtaagtagttcgccagttaatagtttgcgcaacgttgttgccattgctacaggc |
| | | | | | atcgtggtgtcacgctcgtcgtttggtatggcttcattcagctccggttcccaacgatcaaggcgagttacatgatcccccatgttgtgca |
| | | | | | aaaaagcggttagctccttcggtcctccgatcgttgtcagaagtaagttggccgcagtgttatcactcatggttatggcagcactgcata |
| | | | | | attctcttactgtcatgccatccgtaagatgcttttctgtgactggtgagtactcaaccaagtcattctgagaatagtgtatgcggcgacc |
| | | | | | gagttgctcttgcccggcgtcaatacgggataataccgcgccacatagcagaactttaaaagtgctcatcattggaaaacgttcttcgg |
| | | | | | ggcgaaaactctcaaggatcttaccgctgttgagatccagttcgatgtaacccactcgtgcacccaactgatcttcagcatcttttacttt |
| | | | | | caccagcgtttctgggtgagcaaaaacaggaaggcaaaatgccgcaaaaaagggaataagggcgacacggaaatgttgaatactc |
| | | | | | atactcttcctttttcaatcatgattgaagcatttatcagggttattgtctcatgagcggatacatatttgaatgtatttagaaaaataaac |
| | | | | | aaataggtcatgaccaaaatcccttaacgtgagttttcgttccactgagcgtcagaccccgtagaaaagatcaaaggatcttcttgaga |
| | | | | | tcctttttttctgcgcgtaatctgctgcttgcaaacaaaaaaaccaccgctaccagcggtggtttgtttgccggatcaagagctaccaact |
| | | | | | ctttttccgaaggtaactggcttcagcagagcgcagataccaaatactgtccttctagtgtagccgtagttaggccaccacttcaagaac |
| | | | | | tctgtagcaccgcctacatacctcgctctgctaatcctgttaccagtggctgctgccagtggcgataagtcgtgtcttaccgggttggact |
| | | | | | caagacgatagttaccggataaggcgcagcggtcgggctgaacggggggttcgtgcacacagcccagcttggagcgaacgacctac |
| | | | | | accgaactgagatacctacagcgtgagctatgagaaagcgccacgcttcccgaagggagaaaggcggacaggtatccggtaagcgg |
| | | | | | cagggtcggaacaggagagcgcacgagggagcttccagggggaaacgcctggtatctttatagtcctgtcgggtttcgccacctctga |
| | | | | | cttgagcgtcgatttttgtgatgctcgtcaggggggcggagcctatggaaaaacgccagcaacgcggcctttttacggttcctggccttt |
| | | | | | tgctggccttttgctcacatgttctttcctgcgttatcccctgattctgtggataaccgtattaccgcctttgagtgagctgataccgctcgc |
| | | | | | cgcagccgaacgaccgagcgcagcgagtcagtgagcgaggaagcggaagagcgcctgatgcggtattttctccttacgcatctgtgc |
| | | | | | ggtatttcacaccgcatatatggtgcactctcagtacaatctgctctgatgccgcatagttaagccagtatacactccgctatcgctacgt |
| | | | | | gactgggtcatggctgcgccccgacacccgccaacacccgctgacgcgccctgacgggcttgtctgctcccggcatccgcttacagac |
| | | | | | aagctgtgaccgtctccgggagctgcatgtgtcagaggttttcaccgtcatcaccgaaacgcgcgaggcagctgcggtaaagctcatc |
| | | | | | agcgtggtcgtgaagcgattcacagatgtctgcctgttcatccgcgtccagctcgttgagtttctccagaagcgttaatgtctggcttctg |
| | | | | | ataaagcgggccatgttaagggcggttttttcctgtttggtcactgatgcctccgtgtaagggggatttctgttcatgggggtaatgatac |
| | | | | | cgatgaaacgagagaggatgctcacgatacgggttactgatgatgaacatgcccggttactggaacgttgtgagggtaaacaactgg |
| | | | | | cggtatggatgcggcgggaccagagaaaaatcactcagggtcaatgccagcgcttcgttaatacagatgtaggtgttccacagggtag |
| | | | | | ccagcagcatcctgcgatgcagatccggaacataatggtgcagggcgctgacttccgcgtttccagactttacgaaacacggaaaccg |
| | | | | | aagaccattcatgttgttgctcaggtcgcagacgttttgcagcagcagtcgcttcacgttcgctcgcgtatcggtgattcattctgctaac |
| | | | | | cagtaaggcaaccccgccagcctagccgggtcctcaacgacaggagcacgatcatgctagtcatgccccgcgcccaccggaaggag |
| | | | | | ctgactgggttgaaggctctcaagggcatcggtcgagatcccggtgcctaatgagtgagctaacttacattaattgcgttgcgctcactg |
| | | | | | cccgctttccagtcgggaaacctgtcgtgccagctgcattaatgaatcggccaacgcgcggggagaggcggtttgcgtattgggcgcc |
| | | | | | agggtggtttttcttttcaccagtgagacgggcaacagctgattgcccttcaccgcctggccctgagagagttgcagcaagcggtccac |
| | | | | | gctggtttgccccagcaggcgaaaatcctgtttgatggtggttaacggcgggatataacatgagctgtcttcggtatcgtcgtatcccac |
| | | | | | taccgagatgtccgcaccaacgcgcagcccggactcggtaatggcgcgcattgcgcccagcgccatctgatcgttggcaaccagcatc |
| | | | | | gcagtgggaacgatgccctcattcagcatttgcatggtttgttgaaaaccggacatggcactccagtcgccttcccgttccgctatcggc |
| | | | | | tgaatttgattgcgagtgagatatttatgccagccagccagacgcagacgcgccgagacagaacttaatgggcccgctaacagcgcg |
| | | | | | atttgctggtgacccaatgcgaccagatgctccacgcccagtcgcgtaccgtcttcatgggagaaaataatactgttgatgggtgtctg |
| | | | | | gtcagagacatcaagaaataacgccggaacattagtgcaggcagcttccacagcaatggcatcctggtcatccagcggatagttaat |
| | | | | | gatcagcccactgacgcgttgcgcgagaagattgtgcaccgccgctttacaggcttcgacgccgcttcgttctaccatcgacaccacca |
| | | | | | cgctggcacccagttgatcggcgcgagatttaatcgccgcgacaatttgcgacggcgcgtgcagggccagactggaggtggcaacgc |
| | | | | | caatcagcaacgactgtttgcccgccagttgttgtgccacgcggttgggaatgtaattcagctccgccatcgccgcttccactttttcccg |
| | | | | | cgttttcgcagaaacgtggctggcctggttcaccacgcgggaaacggtctgataagagacaccggcatactctgcgacatcgtataac |
| | | | | | gttactggtttcacattcaccaccctgaattgactctcttccgggcgctatcatgccataccgcgaaaggttttgcgccattcgatggtgt |
| | | | | | ccgggatctcgacgctctcccttatgcgactcctgcattaggaagcagcccagtagtaggttgaggccgttgagcaccgccgccgcaa |
| | | | | | ggaatggtgcatgcaaggagatggcgcccaacagtcccccggccacggggcctgccaccatacccacgccgaaacaagcgctcatg |
| | | | | | agcccgaagtggcgagcccgatcttccccatcggtgatgtcggcgatataggcgccagcaaccgcacctgtggcgccggtgatgccgg |
| | | | | | ccacgatgcgtccggcgtagaggatcgagatcgatctcgatcccgcgaaattaatacgactcactata |
| 6 | pETDuet-1 | Synthetic | DNA | Other DNA | ggggaattgtgagcggataacaattcccctctagaaataattttgtttaactttaagaaggagatataccatgggcagcagccatcacc |
| | inter- | | | | atcatcaccacagccaggatccatgaaatacctattgcctacggcagccgctggattgttattactcgcggcccagccggccatggccg |
| | mediate | | | | aattcgagctcggcctgatggtgggcggcgtggtgattgcggtcgacaagcttgtgaaaaaattattattcgcaattcctttagtggtac |
| | plasmid + | | | | ctttctattctcactcggccgaaactgttgaaagttgtttagcaaaatcccatacagaaaattcatttactaacgtctggaaagacgaca |
| | Aβ peptide | | | | aaactttagatcgttacgctaactatgagggctgtctgtggaatgctacaggcgttgtagtttgtactggtgacgaaactcagtgttacg |
| | Aβ33-42 | | | | gtacatgggttcctattgggcttgctatccctgaaaatgagggtggtggctctgagggtggcggttctgagggtggcggttctgagggtg |
| | (phagemid | | | | gcggtactaaacctcctgagtacggtgatacacctattccgggctatacttatatcaaccctctcgacggcacttatccgcctggtactg |
| | AB33-42) | | | | agcaaaaccccgctaatcctaatccttctcttgaggagtctcagcctcttaatactttcatgtttcagaataataggttccgaaataggca |
| | | | | | gggggcattaactgtttatacgggcactgttactcaaggcactgaccccgttaaaacttattaccagtacactcctgtatcatcaaaagc |
| | | | | | catgtatgacgcttactggaacggtaaattcagagactgcgctttccattctggctttaatgaggatttatttgtttgtgaatatcaaggc |
| | | | | | caatcgtctgacctgcctcaacctcctgtcaatgctggcggcggctctggtggtggttctggtggcggctctgagggtggtggctctgag |
| | | | | | ggtggcggttctgagggtggcggctctgagggaggcggttccggtggtggctctggttccggtgattttgattatgaaaagatggcaaa |
| | | | | | cgctaataagggggctatgaccgaaaatgccgatgaaaacgcgctacagtctgacgctaaaggcaaacttgattctgtcgctactgat |
| | | | | | tacggtgctgctatcgatggtttcattggtgacgtttccggccttgctaatggtaatggtgctactggtgattttgctggctctaattccca |
| | | | | | aatggctcaagtcggtgacggtgataattcacctttaatgaataatttccgtcaatatttaccttccctccctcaatcggttgaatgtcgc |
| | | | | | ccttttgtctttggcgctggtaaaccatatgaattttctattgattgtgacaaaataaacttattccgtggtgtctttgcgtttcttttatatg |
| | | | | | ttgccacctttatgtatgtattttctacgtttgctaacatactgcgtaataaggagtcttaagcggccgcataatgcttaagtcgaacaga |
| | | | | | aagtaatcgtattgtacacggccgcataatcgaaattaatacgactcactataggggaattgtgagcggataacaattccccatcttag |
| | | | | | tatattagttaagtataagaaggagatatacatatggcagatctcaattggatatcggccggccacgcgatcgctgacgtcggtaccct |
| | | | | | cgagtctggtaaagaaaccgctgctgcgaaatttgaacgccagcacatggactcgtctactagcgcagcttaattaacctaggctgctg |
| | | | | | ccaccgctgagcaataactagcataaccccttggggcctctaaacgggtcttgaggggttttttgctgaaaggaggaactatatccgga |
| | | | | | ttggcgaatgggacgcgccctgtagcggcgcattaagcgcggcgggtgtggtggttacgcgcagcgtgaccgctacacttgccagcgc |
| | | | | | cctagcgcccgctcctttcgctttcttcccttcctttctcgccacgttcgccggctttccccgtcaagctctaaatcgggggctccctttagg |
| | | | | | gttccgatttagtgctttacggcacctcgaccccaaaaaacttgattagggtgatggttcacgtagtgggccatcgccctgatagacggt |
| | | | | | ttttcgccctttgacgttggagtccacgttctttaatagtggactcttgttccaaactggaacaacactcaaccctatctcggtctattcttt |
| | | | | | tgatttataagggattttgccgatttcggcctattggttaaaaaatgagctgatttaacaaaaatttaacgcgaattttaacaaaatatta |
| | | | | | acgtttacaatttctggcggcacgatggcatgagattatcaaaaaggatcttcacctagatccttttaaattaaaaatgaagttttaaat |
| | | | | | caatctaaagtatatatgagtaaacttggtctgacagttaccaatgcttaatcagtgaggcacctatctcagcgatctgtctatttcgttc |
| | | | | | atccatagttgcctgactccccgtcgtgtagataactacgatacgggagggcttaccatctggccccagtgctgcaatgataccgcgag |
| | | | | | acccacgctcaccggctccagatttatcagcaataaaccagccagccggaagggccgagcgcagaagtggtcctgcaactttatccgc |
| | | | | | ctccatccagtctattaattgttgccgggaagctagagtaagtagttcgccagttaatagtttgcgcaacgttgttgccattgctacaggc |
| | | | | | atcgtggtgtcacgctcgtcgtttggtatggcttcattcagctccggttcccaacgatcaaggcgagttacatgatcccccatgttgtgca |
| | | | | | aaaaagcggttagctccttcggtcctccgatcgttgtcagaagtaagttggccgcagtgttatcactcatggttatggcagcactgcata |
| | | | | | attctcttactgtcatgccatccgtaagatgcttttctgtgactggtgagtactcaaccaagtcattctgagaatagtgtatgcggcgacc |
| | | | | | gagttgctcttgcccggcgtcaatacgggataataccgcgccacatagcagaactttaaaagtgctcatcattggaaaacgttcttcgg |
| | | | | | ggcgaaaactctcaaggatcttaccgctgttgagatccagttcgatgtaacccactcgtgcacccaactgatcttcagcatcttttacttt |
| | | | | | caccagcgtttctgggtgagcaaaaacaggaaggcaaaatgccgcaaaaaagggaataagggcgacacggaaatgttgaatactc |
| | | | | | atactcttcctttttcaatcatgattgaagcatttatcagggttattgtctcatgagcggatacatatttgaatgtatttagaaaaataaac |
| | | | | | aaataggtcatgaccaaaatcccttaacgtgagttttcgttccactgagcgtcagaccccgtagaaaagatcaaaggatcttcttgaga |
| | | | | | tcctttttttctgcgcgtaatctgctgcttgcaaacaaaaaaaccaccgctaccagcggtggtttgtttgccggatcaagagctaccaact |
| | | | | | ctttttccgaaggtaactggcttcagcagagcgcagataccaaatactgtccttctagtgtagccgtagttaggccaccacttcaagaac |
| | | | | | tctgtagcaccgcctacatacctcgctctgctaatcctgttaccagtggctgctgccagtggcgataagtcgtgtcttaccgggttggact |
| | | | | | caagacgatagttaccggataaggcgcagcggtcgggctgaacggggggttcgtgcacacagcccagcttggagcgaacgacctac |
| | | | | | accgaactgagatacctacagcgtgagctatgagaaagcgccacgcttcccgaagggagaaaggcggacaggtatccggtaagcgg |
| | | | | | cagggtcggaacaggagagcgcacgagggagcttccagggggaaacgcctggtatctttatagtcctgtcgggtttcgccacctctga |
| | | | | | cttgagcgtcgatttttgtgatgctcgtcaggggggcggagcctatggaaaaacgccagcaacgcggcctttttacggttcctggccttt |
| | | | | | tgctggccttttgctcacatgttctttcctgcgttatcccctgattctgtggataaccgtattaccgcctttgagtgagctgataccgctcgc |
| | | | | | cgcagccgaacgaccgagcgcagcgagtcagtgagcgaggaagcggaagagcgcctgatgcggtattttctccttacgcatctgtgc |
| | | | | | ggtatttcacaccgcatatatggtgcactctcagtacaatctgctctgatgccgcatagttaagccagtatacactccgctatcgctacgt |
| | | | | | gactgggtcatggctgcgccccgacacccgccaacacccgctgacgcgccctgacgggcttgtctgctcccggcatccgcttacagac |
| | | | | | aagctgtgaccgtctccgggagctgcatgtgtcagaggttttcaccgtcatcaccgaaacgcgcgaggcagctgcggtaaagctcatc |
| | | | | | agcgtggtcgtgaagcgattcacagatgtctgcctgttcatccgcgtccagctcgttgagtttctccagaagcgttaatgtctggcttctg |
| | | | | | ataaagcgggccatgttaagggcggttttttcctgtttggtcactgatgcctccgtgtaagggggatttctgttcatgggggtaatgatac |
| | | | | | cgatgaaacgagagaggatgctcacgatacgggttactgatgatgaacatgcccggttactggaacgttgtgagggtaaacaactgg |
| | | | | | cggtatggatgcggcgggaccagagaaaaatcactcagggtcaatgccagcgcttcgttaatacagatgtaggtgttccacagggtag |
| | | | | | ccagcagcatcctgcgatgcagatccggaacataatggtgcagggcgctgacttccgcgtttccagactttacgaaacacggaaaccg |
| | | | | | aagaccattcatgttgttgctcaggtcgcagacgttttgcagcagcagtcgcttcacgttcgctcgcgtatcggtgattcattctgctaac |
| | | | | | cagtaaggcaaccccgccagcctagccgggtcctcaacgacaggagcacgatcatgctagtcatgccccgcgcccaccggaaggag |
| | | | | | ctgactgggttgaaggctctcaagggcatcggtcgagatcccggtgcctaatgagtgagctaacttacattaattgcgttgcgctcactg |
| | | | | | cccgctttccagtcgggaaacctgtcgtgccagctgcattaatgaatcggccaacgcgcggggagaggcggtttgcgtattgggcgcc |
| | | | | | agggtggtttttcttttcaccagtgagacgggcaacagctgattgcccttcaccgcctggccctgagagagttgcagcaagcggtccac |
| | | | | | gctggtttgccccagcaggcgaaaatcctgtttgatggtggttaacggcgggatataacatgagctgtcttcggtatcgtcgtatcccac |
| | | | | | taccgagatgtccgcaccaacgcgcagcccggactcggtaatggcgcgcattgcgcccagcgccatctgatcgttggcaaccagcatc |
| | | | | | gcagtgggaacgatgccctcattcagcatttgcatggtttgttgaaaaccggacatggcactccagtcgccttcccgttccgctatcggc |
| | | | | | tgaatttgattgcgagtgagatatttatgccagccagccagacgcagacgcgccgagacagaacttaatgggcccgctaacagcgcg |
| | | | | | atttgctggtgacccaatgcgaccagatgctccacgcccagtcgcgtaccgtcttcatgggagaaaataatactgttgatgggtgtctg |
| | | | | | gtcagagacatcaagaaataacgccggaacattagtgcaggcagcttccacagcaatggcatcctggtcatccagcggatagttaat |
| | | | | | gatcagcccactgacgcgttgcgcgagaagattgtgcaccgccgctttacaggcttcgacgccgcttcgttctaccatcgacaccacca |
| | | | | | cgctggcacccagttgatcggcgcgagatttaatcgccgcgacaatttgcgacggcgcgtgcagggccagactggaggtggcaacgc |
| | | | | | caatcagcaacgactgtttgcccgccagttgttgtgccacgcggttgggaatgtaattcagctccgccatcgccgcttccactttttcccg |
| | | | | | cgttttcgcagaaacgtggctggcctggttcaccacgcgggaaacggtctgataagagacaccggcatactctgcgacatcgtataac |
| | | | | | gttactggtttcacattcaccaccctgaattgactctcttccgggcgctatcatgccataccgcgaaaggttttgcgccattcgatggtgt |
| | | | | | ccgggatctcgacgctctcccttatgcgactcctgcattaggaagcagcccagtagtaggttgaggccgttgagcaccgccgccgcaa |
| | | | | | ggaatggtgcatgcaaggagatggcgcccaacagtcccccggccacggggcctgccaccatacccacgccgaaacaagcgctcatg |
| | | | | | agcccgaagtggcgagcccgatcttccccatcggtgatgtcggcgatataggcgccagcaaccgcacctgtggcgccggtgatgccgg |
| | | | | | ccacgatgcgtccggcgtagaggatcgagatcgatctcgatcccgcgaaattaatacgactcactata |
| 7 | Primer | Synthetic | DNA | Other DNA | ccctcatagttagcgtaacg |
| 8 | Recomb- | Synthetic | Amino | Protein | MDAEFRHDSGYEVHHQKLVFFAEDVGSNKGAIIGLMVGGVVIA |
| | inant | | acid | | |
| | Aβ42 | | | | |
| 9 | Plasmid | Synthetic | DNA | Other DNA | atcagttgggtgcacgagtgggttacatcgaactggatctcaacagcggtaagatccttg |
| | PET- | | | | agagttttcgccccgaagaacgttttccaatgatgagcacttttaaagttctgctatgtggcgcggtattatcccgtgttgacgccgggc |
| | Sac-Abeta | | | | aagagcaactcggtcgccgcatacactattctcagaatgacttggttgagtactcaccagtcacagaaaagcatcttacggatggcat |
| | (M1-42) | | | | gacagtaagagaattatgcagtgctgccataaccatgagtgataacactgcggccaacttacttctgacaacgatcggaggaccgaa |
| | | | | | ggagctaaccgcttttttgcacaacatgggggatcatgtaactcgccttgatcgttgggaaccggagctgaatgaagccataccaaac |
| | | | | | gacgagcgtgacaccacgatgcctgcagcaatggcaacaacgttgcgcaaactattaactggcgaactacttactctagcttcccggc |
| | | | | | aacaattaatagactggatggaggcggataaagttgcaggaccacttctgcgctcggcccttccggctggctggtttattgctgataaa |
| | | | | | tctggagccggtgagcgtgggtctcgcggtatcattgcagcactggggccagatggtaagccctcccgtatcgtagttatctacacgac |
| | | | | | ggggagtcaggcaactatggatgaacgaaatagacagatcgctgagataggtgcctcactgattaagcattggtaactgtcagacca |
| | | | | | agtttactcatatatactttagattgatttaaaacttcatttttaatttaaaaggatctaggtgaagatcctttttgataatctcatgaccaa |
| | | | | | aatcccttaacgtgagttttcgttccactgagcgtcagaccccgtagaaaagatcaaaggatcttcttgagatcctttttttctgcgcgta |
| | | | | | atctgctgcttgcaaacaaaaaaaccaccgctaccagcggtggtttgtttgccggatcaagagctaccaactctttttccgaaggtaact |
| | | | | | ggcttcagcagagcgcagataccaaatactgtccttctagtgtagccgtagttaggccaccacttcaagaactctgtagcaccgcctac |
| | | | | | atacctcgctctgctaatcctgttaccagtggctgctgccagtggcgataagtcgtgtcttaccgggttggactcaagacgatagttacc |
| | | | | | ggataaggcgcagcggtcgggctgaacggggggttcgtgcacacagcccagcttggagcgaacgacctacaccgaactgagatacc |
| | | | | | tacagcgtgagctatgagaaagcgccacgcttcccgaagggagaaaggcggacaggtatccggtaagcggcagggtcggaacagg |
| | | | | | agagcgcacgagggagcttccagggggaaacgcctggtatctttatagtcctgtcgggtttcgccacctctgacttgagcgtcgattttt |
| | | | | | gtgatgctcgtcaggggggcggagcctatggaaaaacgccagcaacgcggcctttttacggttcctggccttttgctggccttttgctca |
| | | | | | catgttctttcctgcgttatcccctgattctgtggataaccgtattaccgcctttgagtgagctgataccgctcgccgcagccgaacgacc |
| | | | | | gagcgcagcgagtcagtgagcgaggaagcggaagagcgcctgatgcggtattttctccttacgcatctgtgcggtatttcacaccgca |
| | | | | | atggtgcactctcagtacaatctgctctgatgccgcatagttaagccagtatacactccgctatcgctacgtgactgggtcatggctgcg |
| | | | | | ccccgacacccgccaacacccgctgacgcgccctgacgggcttgtctgctcccggcatccgcttacagacaagctgtgaccgtctccgg |
| | | | | | gagctgcatgtgtcagaggttttcaccgtcatcaccgaaacgcgcgaggcagctgcggtaaagctcatcagcgtggtcgtgaagcgat |
| | | | | | tcacagatgtctgcctgttcatccgcgtccagctcgttgagtttctccagaagcgttaatgtctggcttctgataaagcgggccatgttaa |
| | | | | | gggcggttttttcctgtttggtcactgatgcctccgtgtaagggggatttctgttcatgggggtaatgataccgatgaaacgagagagga |
| | | | | | tgctcacgatacgggttactgatgatgaacatgcccggttactggaacgttgtgagggtaaacaactggcggtatggatgcggcggga |
| | | | | | ccagagaaaaatcactcagggtcaatgccagcgcttcgttaatacagatgtaggtgttccacagggtagccagcagcatcctgcgatg |
| | | | | | cagatccggaacataatggtgcagggcgctgacttccgcgtttccagactttacgaaacacggaaaccgaagaccattcatgttgttgc |
| | | | | | tcaggtcgcagacgttttgcagcagcagtcgcttcacgttcgctcgcgtatcggtgattcattctgctaaccagtaaggcaaccccgcca |
| | | | | | gcctagccgggtcctcaacgacaggagcacgatcatgcgcacccgtggccaggacccaacgctgcccgagatgcgccgcgtgcggct |
| | | | | | gctggagatggcggacgcgatggatatgttctgccaagggttggtttgcgcattcacagttctccgcaagaattgattggctccaattct |
| | | | | | tggagtggtgaatccgttagcgaggtgccgccggcttccattcaggtcgaggtggcccggctccatgcaccgcgacgcaacgcgggga |
| | | | | | ggcagacaaggtatagggggcgcctacaatccatgccaacccgttccatgtgctcgccgaggcggcataaatcgccgtgacgatca |
| | | | | | gcggtccaatgatcgaagttaggctggtaagagccgcgagcgatccttgaagctgtccctgatggtcgtcatctacctgcctggacagc |
| | | | | | atggcctgcaacgcgggcatcccgatgccgccggaagcgagaagaatcataatggggaaggccatccagcctcgcgtcgcgaacgc |
| | | | | | cagcaagacgtagcccagcgcgtcggccgccatgccggcgataatggcctgcttctcgccgaaacgtttggtgggggaccagtgacg |
| | | | | | aaggcttgagcgagggcgtgcaagattccgaataccgcaagcgacaggccgatcatcgtcgcgctccagcgaaagcggtcctcgccg |
| | | | | | aaaatgacccagagcgctgccggcacctgtcctacgagttgcatgataaagaagacagtcataagtgcggcgacgatagtcatgccc |
| | | | | | cgcgcccaccggaaggagctgactgggttgaaggctctcaagggcatcggtcgacgctctcccttatgcgactcctgcattaggaagc |
| | | | | | agcccagtagtaggttgaggccgttgagcaccgccgccgcaaggaatggtgcatgcaaggagatggcgcccaacagtcccccggcc |
| | | | | | acggggcctgccaccatacccacgccgaaacaagcgctcatgagcccgaagtggcgagcccgatcttccccatcggtgatgtcggcg |
| | | | | | atataggcgccagcaaccgcacctgtggcgccggtgatgccggccacgatgcgtccggcgtagaggatcgagatctcgatcccgcga |
| | | | | | aattaatacgactcactatagggagaccacaacggtttccctctagaaataattttgtttaactttaagaaggagatatacatatggac |
| | | | | | gctgaattccgtcacgactctggttacgaagttcaccaccagaagctggtgttcttcgctgaagacgtgggttctaacaagggtgctatc |
| | | | | | atcggtctgatggttggtggcgttgtgatcgcttaataggagctcgatccggctgctaacaaagcccgaaaggaagctgagttggctgc |
| | | | | | tgccaccgctgagcaataactagcataaccccttggggcctctaaacgggtcttgaggggttttttgctgaaaggaggaactatatccg |
| | | | | | gatatccacaggacgggtgtggtcgccatgatcgcgtagtcgatagtggctccaagtagcgaagcgagcaggactgggcggcggcca |
| | | | | | aagcggtcggacagtgctccgagaacgggtgcgcatagaaattgcatcaacgcatatagcgctagcagcacgccatagtgactggcg |
| | | | | | atgctgtcggaatggacgatatcccgcaagaggcccggcagtaccggcataaccaagcctatgcctacagcatccagggtgacggtg |
| | | | | | ccgaggatgacgatgagcgcattgttagatttcatacacggtgcctgactgcgttagcaatttaactgtgataaactaccgcattaaag |
| | | | | | cttatcgatgataagctgtcaaacatgagaattcttgaagacgaaagggcctcgtgatacgcctatttttataggttaatgtcatgataa |
| | | | | | taatggtttcttagacgtcaggtggcacttttcggggaaatgtgcgcggaacccctatttgtttatttttctaaatacattcaaatatgtat |
| | | | | | ccgctcatgagacaataaccctgataaatgcttcaataatattgaaaaaggaagagtatgagtattcaacatttccgtgtcgcccttatt |
| | | | | | cccttttttgcggcattttgccttcctgtttttgctcacccagaaacgctggtgaaagtaaaagatgctgaag |
| 10 | Aβ36-39 | Synthetic | Aminoacid | Protein | VGGV |
| 11 | Aβ36-39- | Synthetic | DNA | Other DNA | gtgggcggcgtg |
| | nucleotide | | | | |
| | sequence | | | | |
| 12 | Aβ30-39- | Synthetic | DNA | Other DNA | gcgattattggcctgatggtgggcggcgtg |
| | nucleotide | | | | |
| | sequence | | | | |
| 13 | Aβ33-42- | Synthetic | DNA | Other DNA | ggcctgatggtgggcggcgtggtgattgcg |
| | nucleotide | | | | |
| | sequence | | | | |

### REFERENCES

1. Kamenetz F, et al. APP processing and synaptic function. Neuron 37, 925-937 (2003).
2. Hardy J, Selkoe D J. The amyloid hypothesis of Alzheimer's disease: progress and problems on the road to therapeutics. Science 297, 353-356 (2002).
3. Huang Y R, Liu R T. The Toxicity and Polymorphism of beta-Amyloid Oligomers. International Journal of Molecular Sciences 21, (2020).
4. Mucke L, et al. High-level neuronal expression of abeta 1-42 in wild-type human amyloid protein precursor transgenic mice: synaptotoxicity without plaque formation. The Journal of neuroscience: the official journal of the Society for Neuroscience 20, 4050-4058 (2000).
5. Jokar S, et al. Amyloid beta-Targeted Inhibitory Peptides for Alzheimer's Disease: Current State and Future Perspectives. In: Alzheimer's Disease: Drug Discovery (ed Huang X) (2020).
6. Farr R, Choi D S, Lee S W. Phage-based nanomaterials for biomedical applications. Acta Biomaterialia 10, 1741-1750 (2014).
7. Schmidt C. Phage therapy's latest makeover. Nature Biotechnology 37, 581-586 (2019).
8. Popescu M, Van Belleghem J D, Khosravi A, Bollyky P L. Bacteriophages and the Immune System. Annual Review of Virology 8, 415-435 (2021).
9. Barr J J. A bacteriophages journey through the human body. Immunological Reviews 279, 106-122 (2017).
10. Ghose C, et al. The Virome of Cerebrospinal Fluid: Viruses Where We Once Thought There Were None. Frontiers in Microbiology 10, 2061 (2019).
11. Perchiacca J M, Ladiwala A R, Bhattacharya M, Tessier P M. Structure-based design of conformation- and sequence-specific antibodies against amyloid beta. Proceedings of the National Academy of Sciences of the United States of America 109, 84-89 (2012).
12. Walsh D M, et al. A facile method for expression and purification of the Alzheimer's disease-associated amyloid beta-peptide. The FEBS Journal 276, 1266-1281 (2009).
13. Qi H, Lu H, Qiu H J, Petrenko V, Liu A. Phagemid vectors for phage display: properties, characteristics and construction. Journal of Molecular Biology 417, 129-143 (2012).
14. Needleman S B and Wunsch C D. A general method applicable to the search for similarities in the amino acid sequence of two proteins. Journal of Molecular Biology 48, 443-453 (1970).
15. Altschul S F, Gish W, Miller W, Myers E W, Lipman D J. Basic local alignment search tool. Journal of Molecular Biology 215, 403-410 (1990).
16. Campanella J J, Bitinka L, Smalley J. MatGAT: an application that generates similarity/identity matrices using protein or DNA sequences. BMC Bioinformatics 4:29 (2003).
17. Cohen S I, et al. Proliferation of amyloid-beta42 aggregates occurs through a secondary nucleation mechanism. Proceedings of the National Academy of Sciences of the United States of America 110, 9758-9763 (2013).
18. Tornquist M, et al. Secondary nucleation in amyloid formation. Chemical Communications 54, 8667-8684 (2018).
19. Gremer L, et al. Fibril structure of amyloid-beta (1-42) by cryo-electron microscopy. Science 358, 116-119 (2017).
20. Trinchese F, Liu S, Battaglia F, Walter S, Mathews P M, Arancio O. Progressive age-related development of Alzheimer-like pathology in APP/PS1 mice. Annals of Neurology 55, 801-814 (2004).
21. Nelson P T, et al. Correlation of Alzheimer disease neuropathologic changes with cognitive status: a review of the literature. Journal of neuropathology and experimental neurology 71, 362-381 (2012).
22. Levenson J M, et al. NPT088 reduces both amyloid-beta and tau pathologies in transgenic mice. Alzheimer's & Dementia (N Y) 2, 141-155 (2016).

1. An engineered M13 bacteriophage comprising, at its surface, amyloidogenic peptide motifs, wherein the peptide motifs comprise at least a sequence 90% identical to the sequences of the following list: SEQ ID No 1, SEQ ID No 2, and mixtures thereof.

2. The bacteriophage of claim 1, wherein the peptide motifs comprise at least a sequence 95% identical to the sequences of the following list: SEQ ID No 1, SEQ ID No 2, and mixtures thereof.

3. The bacteriophage of claim 1, wherein the peptide motifs comprise at least a sequence 100% identical to the sequences of the following list: SEQ ID No 1, SEQ ID No 2, and mixtures thereof.

4. The bacteriophage of claim 1, wherein the peptide motifs comprises at least a sequence 90% identical to SEQ ID No. 1 and a DNA sequence at least 90% identical to SEQ ID No. 5.

5. The bacteriophage of claim 1, wherein the peptide motifs comprises at least a sequence 90% identical to SEQ ID No. 2 and a DNA sequence at least 90% identical to SEQ ID No. 6.

6. The bacteriophage of claim 1, wherein the bacteriophage comprises, at its surface, five to eight of said amyloidogenic peptide motifs.

7. (canceled)

8. (canceled)

9. (canceled)

10. (canceled)

11. (canceled)

12. (canceled)

13. (canceled)

14. The bacteriophage of claim 1, wherein the peptide motifs comprise at least a sequence 100% identical to the sequences of the following list: SEQ ID No 1, SEQ ID No 2, and mixtures thereof.

15. A pharmaceutical composition comprising a bacteriophage of claim 1.

16. The pharmaceutical composition of claim 15, further comprising a suitable pharmaceutical excipient.

17. (canceled)

18. The pharmaceutical composition of claim 15, wherein the intravenous dosage amount is less than 10¹⁰ pfu/day.

19. The pharmaceutical composition of claim 15, wherein the intravenous dosage amount is from 10⁴-10¹⁰ pfu/day.

20. (canceled)

21. A method for treating or preventing Alzheimer's disease or a neurodegenerative disease that is positively influenced by the decrease in aggregation of amyloid-beta oligomers and amyloid beta fibrils in a subject, the method comprising administering the bacteriophage of claim 1 to the subject.

22. A kit for detecting, quantifying, and/or diagnosing amyloid-beta oligomers and/or amyloid-beta fibrils in tissue samples, comprising the bacteriophage claim 1.

23. A method of detecting or diagnosing the presence of amyloid-beta oligomers and/or amyloid-beta fibrils, in a tissue sample comprising the steps of:
incubating the tissue sample with a solution comprising the bacteriophage of claim 1; and
detecting the presence of the bacteriophage in the tissue sample.

24. The method of claim 23, further comprising a step of incubating the tissue sample with a first antibody suitable to bind to the bacteriophage.

25. The method of claim 24, wherein the first antibody is a rabbit anti-fd phage antibody.

26. The method of claim 24, further comprising a step of incubating the tissue sample with a second antibody suitable to bind to the first antibody, wherein the second antibody is a fluorescent antibody.

27. The method of claim 26, wherein the second antibody is a fluorescein isothiocyanate labeled goat anti-rabbit IgG antibody.

28. The method of claim 23, wherein the tissue sample is a brain tissue sample.

29. The method of claim 23, wherein the concentration of bacteriophage in the solution ranges from 10⁴ pfu/ml-10¹⁰ pfu/ml.

30. (canceled)


SEQUENCE LISTING (13 sequences)

SEQ ID NO: 1 - protein, 10 aa, Synthetic construct
AIIGLMVGGV

SEQ ID NO: 2 - protein, 10 aa, synthetic construct
GLMVGGVVIA

SEQ ID NO: 3 - DNA, 5420 bp, synthetic construct
  source 1..5420: mol_type = other DNA
ggggaattgtgagcggataacaattcccctctagaaataattttgtttaactttaagaaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 4 - DNA, 6761 bp, synthetic construct
  source 1..6761: mol_type = other DNA
ggggaattgtgagcggataacaattcccctctagaaataattttgtttaactttaagaaggagatataccatgggcagcagccatcaccatcatcaccacagccaggatccatgaaatacctattgcctacggcagccgctggattgttattactcgcggcccagccggccatggccgaattcgagctcggcgcgcctgcaggtcgacaagcttgtgaaaaaattattattcgcaattcctttagtggtacctttctattctcactcggccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 5 - DNA, 6778 bp, synthetic construct
  source 1..6778: mol_type = other DNA
ggggaattgtgagcggataacaattcccctctagaaataattttgtttaactttaagaaggagatataccatgggcagcagccatcaccatcatcaccacagccaggatccatgaaatacctattgcctacggcagccgctggattgttattactcgcggcccagccggccatggccgaattcgagctcgcgattattggcctgatggtgggcggcgtggtcgacaagcttgtgaaaaaattattattcgcaattcctttagtggtacctttctattctcactcggccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 6 - DNA, 6778 bp, synthetic construct
  source 1..6778: mol_type = other DNA
ggggaattgtgagcggataacaattcccctctagaaataattttgtttaactttaagaaggagatataccatgggcagcagccatcaccatcatcaccacagccaggatccatgaaatacctattgcctacggcagccgctggattgttattactcgcggcccagccggccatggccgaattcgagctcggcctgatggtgggcggcgtggtgattgcggtcgacaagcttgtgaaaaaattattattcgcaattcctttagtggtacctttctattctcactcggccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 7 - DNA, 20 bp, synthetic construct
  source 1..20: mol_type = other DNA
ccctcatagttagcgtaacg

SEQ ID NO: 8 - protein, 43 aa, synthetic construct
MDAEFRHDSGYEVHHQKLVFFAEDVGSNKGAIIGLMVGGVVIA

SEQ ID NO: 9 - DNA, 4741 bp, synthetic construct
  source 1..4741: mol_type = other DNA
atcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggacgctgaattccgtcacgactctggttacgaagttcaccaccagaagctggtgttcttcgctgaagacgtgggttctaacaagggtgctatcatcggtctgatggttggtggcgttgtgatcgcttaataggagctcgatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaag

SEQ ID NO: 10 - protein, 4 aa, synthetic construct
VGGV

SEQ ID NO: 11 - DNA, 12 bp, synthetic construct
  source 1..12: mol_type = other DNA
gtgggcggcgtg

SEQ ID NO: 12 - DNA, 30 bp, synthetic construct
  source 1..30: mol_type = other DNA
gcgattattggcctgatggtgggcggcgtg

SEQ ID NO: 13 - DNA, 30 bp, synthetic construct
  source 1..30: mol_type = other DNA
ggcctgatggtgggcggcgtggtgattgcg